(19) United States
(12) Patent Application Publication
(10) Pub. No.: US 2025/0092003 A1
(43) Pub. Date: Mar. 20, 2025

(54) LIPID COMPOUND, COMPOSITION CONTAINING SAME AND USE

(71) Applicant: SOUTH CHINA UNIVERSITY OF TECHNOLOGY, Guangzhou (CN)

(72) Inventors: Yuan ZHANG, Guangzhou (CN); Fei GU, Guangzhou (CN)

(73) Assignee: SOUTH CHINA UNIVERSITY OF TECHNOLOGY, Guangzhou (CN)

(21) Appl. No.: 18/728,896

(22) PCT Filed: Dec. 2, 2022
(86) PCT No.: PCT/CN2022/136397

§ 371 Date: Jul. 15, 2024

(30) Foreign Application Priority Data
Jan. 14, 2022 (CN) 202210043332.2

(51) Int. Cl. C07D 295/13; A61K 47/14; A61K 47/22; C07C 219/06

(52) U.S. Cl. CPC C07D 295/13; A61K 47/14; A61K 47/22; C07C 219/06

(57) ABSTRACT

Provided are a lipid compound, a composition containing the same and use. The lipid compound is prepared by a ring-opening reaction of an organic amine and glycidyl ester and does not structurally contain free amino. The lipid compound can be ionized into a cationic compound under acidic conditions and is bonded to a negatively charged active pharmaceutical ingredient through electrostatic interaction, thereby being assembled into a drug-loaded lipid nanoparticle for delivery of the active pharmaceutical ingredient. The lipid compound provided by the present invention has a simple structure, a simple reaction path and a high yield. The constructed drug-loaded lipid nanoparticle can be used for preparing nucleic acid drugs, genetic vaccines, polypeptide or protein drugs, and small molecule drugs, and has a broad application prospect.

### TECHNICAL FIELD

The present invention belongs to the technical field of biology and specifically relates to a lipid compound, a composition containing the same and use.

### DESCRIPTION OF RELATED ART

Nucleic acid drugs, by specifically up-regulating or down-regulating the gene expression to correct, knock out or compensate for gene defects or abnormalities, are used for treating genetic diseases, cancer, infectious diseases, autoimmune diseases and cardiovascular diseases. A variety of methods for treating diseases regarding genes have also been promoted in clinical application to bring new hope for medical treatment and health of humans. Common nucleic acid drugs mainly include a plasmid DNA (pDNA), a messenger RNA (mRNA), a small interfering RNA (siRNA) and an antisense oligonucleotide. The siRNA is a double-stranded small molecule RNA that is generally composed of 19 to 25 nucleotides. The siRNA can specifically recognize a target sequence, bind to the mRNA having a complementary sequence and promote degradation of the mRNA, thereby inhibiting the gene expression at a transcriptional level, inducing deletion of specific genes in cells, efficiently silencing pathogenic genes and preventing the occurrence of diseases. Based on the principle of using RNA interference, the idea of using the siRNA as a gene drug has been widely concerned after being put forward and has a broad development prospect.

Compared with traditional chemical drugs and antibody drugs, the nucleic acid drugs have the characteristics of high efficacy, high specificity, low side effects and low risk, and have a relatively simple development process. However, the development process of the nucleic acid drugs still has various “bottleneck” technical problems. First, nucleic acid molecules, such as RNAs, are sensitive to enzymes and are easily degraded by ubiquitous RNA enzymes, thus losing drug activity effects. Second, the nucleic acid drugs entering the body need to undergo complex processes, such as uptake by cells, escape out of endosomes, etc., and are then released to specific sites to exert biological functions. Therefore, the research and development of efficient and safe delivery systems are among the primary tasks to address the challenges in the development problems of nucleic acid drugs.

At present, there are mainly two kinds of technical means for efficient transfection of the nucleic acid drugs: (1) viral vectors, which offer high transfection efficiency but come with potential risks and limited targetability due to the size constraints of the carried genes; and (2) non-viral vectors, including inorganic materials, polymer molecules, liposomes, etc., which have lower transfection efficiency compared to viral vectors. The inorganic materials are difficult to metabolize in the body, and have poor biocompatibility and certain safety problems, while the liposomes and the polymer molecules have low biological toxicity. Compared to liposomes, exogenously synthesized polymer molecules are more likely to induce immunogenicity. Therefore, liposomes have become the most ideal non-viral genetic vector materials for the delivery of nucleic acid drugs. In addition, it has been reported that although nanoparticles have good uptake by cells, only 2% of the nanoparticles can escape out of the endosomes and then reach the cytoplasm to exert physiological functions. Lipid/drug complexes can be formed through electrostatic interaction of cationic lipids carried with positive charges and nucleic acid molecules or protein molecules carried with negative charges, then enter the cytoplasm through endocytosis of cells and transfer to the endosomes. The positively charged lipids can fuse with endosome membranes and release drugs and other contents coated by lipid nanoparticles into the cytoplasm, so as to realize escape out of the endosomes. Although the cationic liposomes have become one of the most widely used non-viral vectors and have good biosafety, the cationic liposomes still have relatively low transfection efficiency at present.

### SUMMARY

The present invention aims to solve the technical problems of the prior art. For this reason, the present invention provides a lipid compound, a composition containing the same and use. The lipid compound has a simple structure, a simple reaction path and a high yield. In addition, the composition constructed by the lipid compound can efficiently deliver an active pharmaceutical ingredient to cells or tissues and has a wide application prospect.

In a first aspect of the present invention, a lipid compound is provided, where the lipid compound is obtained by substituting all hydrogen atoms on an organic amine nitrogen with an R₁ group; the organic amine is selected from structures shown below:

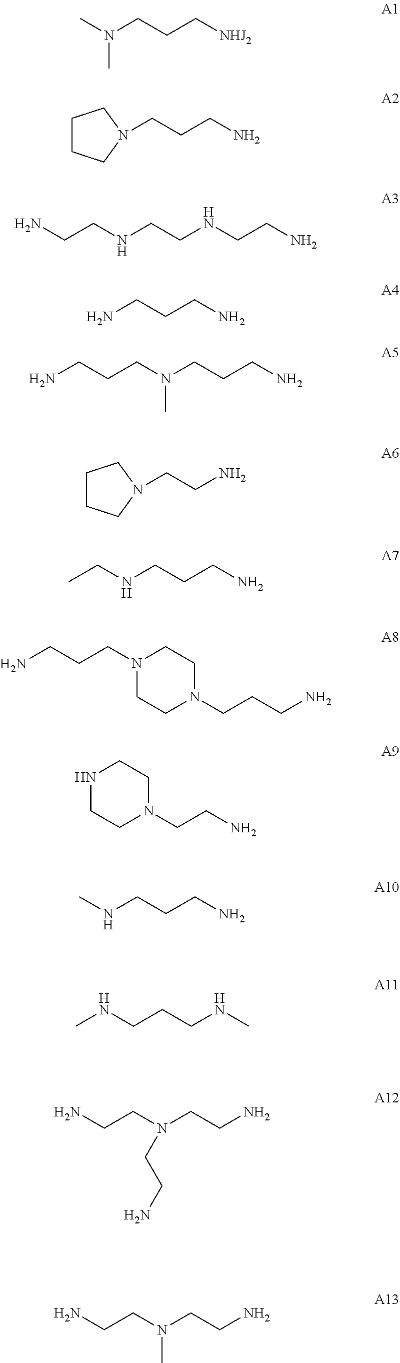

and the R₁ group has a structure shown in Formula (I):

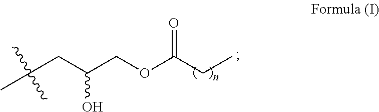

where, n is any integer ranging from 6 to 16.

In some embodiments of the present invention, the R₁ group is selected from structures shown below:

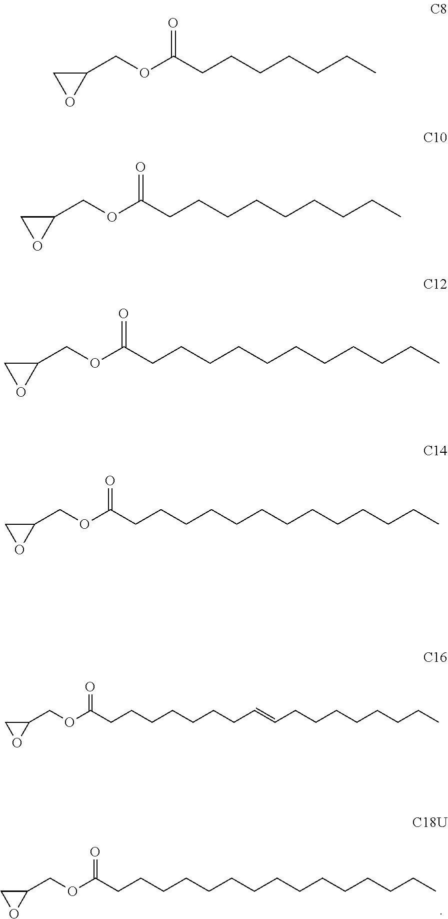 [SMILES: CCCCCCCC(=O)OCC1CO1 ; CCCCCCCCCC(=O)OCC1CO1 ; CCCCCCCCCCCC(=O)OCC1CO1 ; CCCCCCCCCCCCCC(=O)OCC1CO1 ; CCCCCCCC/C=C/CCCCCCCC(=O)OCC1CO1 ; CCCCCCCCCCCCCCCC(=O)OCC1CO1]

In some preferred embodiments of the present invention, the organic amine is selected from A1, A2, A7, A8, A12, and A13.

In some preferred embodiments of the present invention, the R₁ group is selected from C12, C16, and C18U.

In some embodiments of the present invention, the lipid compound is structurally free of free amino.

In some embodiments of the present invention, the lipid compound is selected from structures shown below:

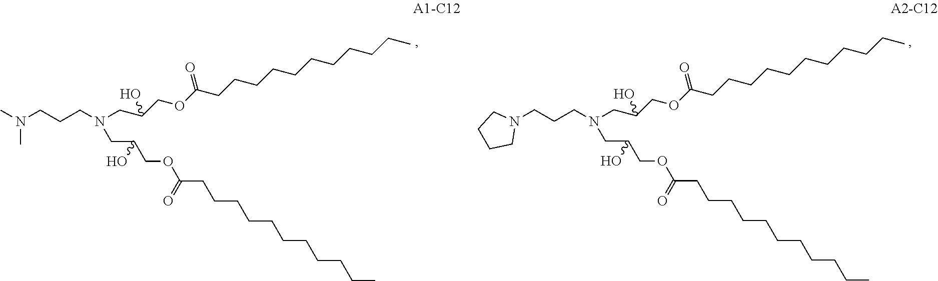 [SMILES: CCCCCCCCCCCC(=O)OCC(O)CN(CCCN(C)C)CC(O)COC(=O)CCCCCCCCCCC ; CCCCCCCCCCCC(=O)OCC(O)CN(CCCN1CCCC1)CC(O)COC(=O)CCCCCCCCCCC]

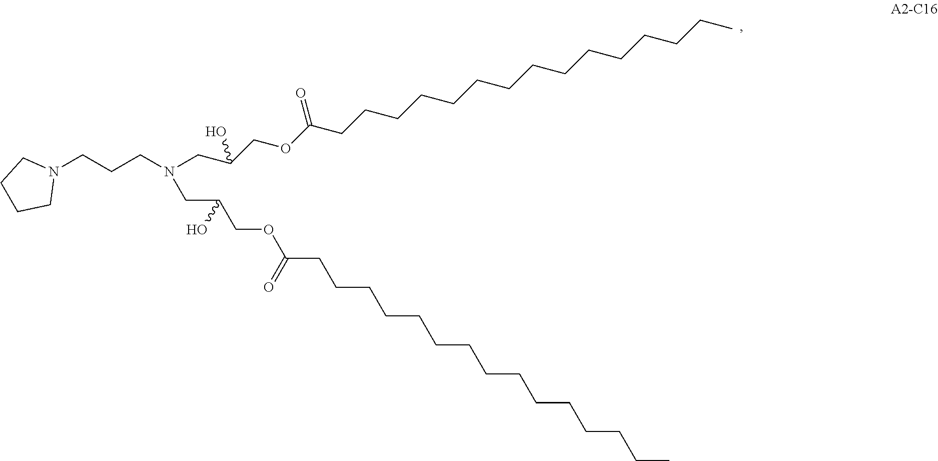 [SMILES: CCCCCCCCCCCCCCCC(=O)OCC(O)CN(CCCN1CCCC1)CC(O)COC(=O)CCCCCCCCCCCCCCC]

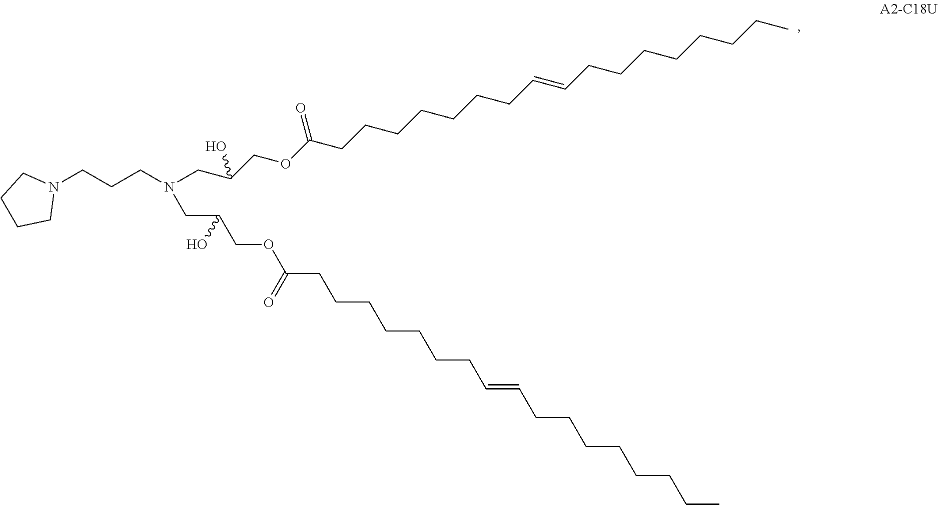 [SMILES: CCCCCCCC/C=C/CCCCCCCC(=O)OCC(O)CN(CCCN1CCCC1)CC(O)COC(=O)CCCCCCC/C=C/CCCCCCCC]

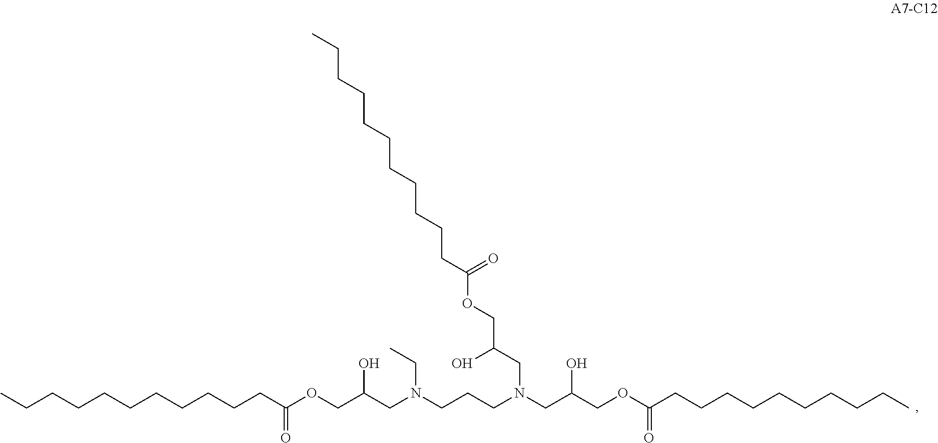 [SMILES: CCCCCCCCCCCC(=O)OCC(O)CN(CC)CCCN(CC(O)COC(=O)CCCCCCCCCC)CC(O)COC(=O)CCCCCCCCCCC]

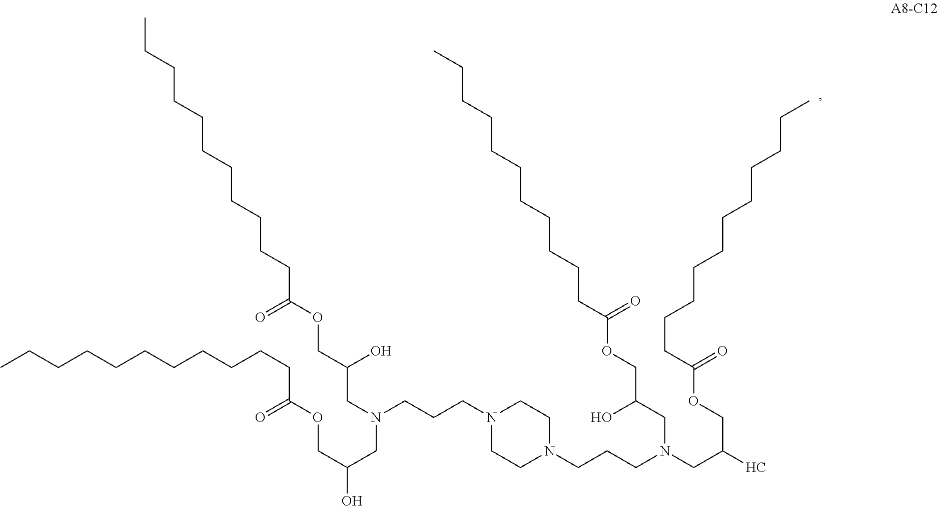 [SMILES: CCCCCCCCCCCC(=O)OCC(C)CN(CCCN1CCN(CCCN(CC(O)COC(=O)CCCCCCCCCCC)CC(O)COC(=O)CCCCCCCCCCC)CC1)CC(O)COC(=O)CCCCCCCCCCC]

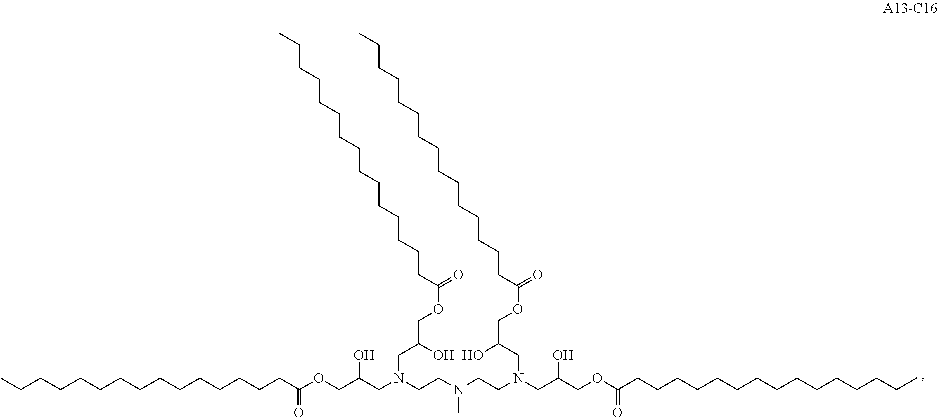 [SMILES: CCCCCCCCCCCCCCCC(=O)OCC(O)CN(CCN(C)CCN(CC(O)COC(=O)CCCCCCCCCCCCCCC)CC(O)COC(=O)CCCCCCCCCCCCCCC)CC(O)COC(=O)CCCCCCCCCCCCCCC]

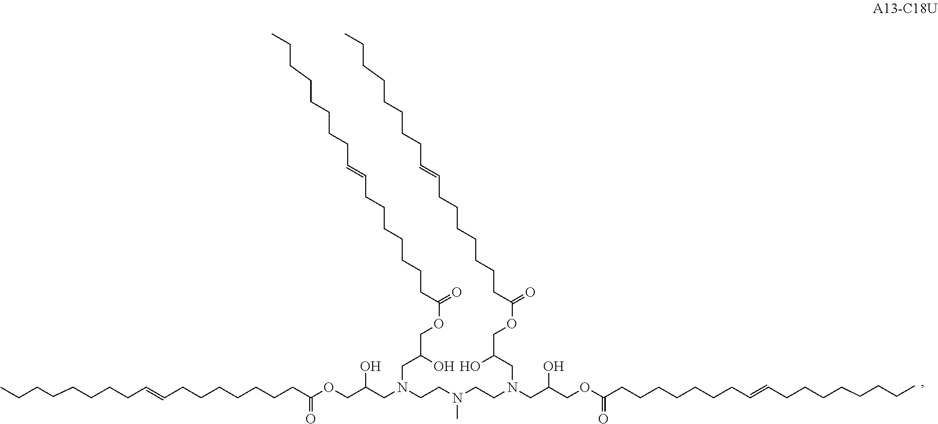 [SMILES: CCCCCCCC/C=C/CCCCCCCC(=O)OCC(O)CN(CCN(C)CCN(CC(O)COC(=O)CCCCCCC/C=C/CCCCCCCC)CC(O)COC(=O)CCCCCCC/C=C/CCCCCCCC)CC(O)COC(=O)CCCCCCC/C=C/CCCCCCCC]

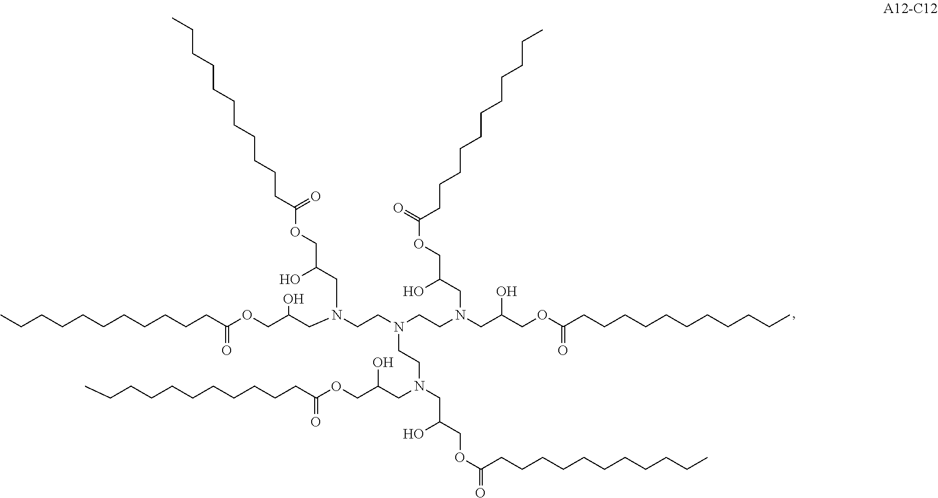 [SMILES: CCCCCCCCCCCC(=O)OCC(O)CN(CCN(CCN(CC(O)COC(=O)CCCCCCCCCCC)CC(O)COC(=O)CCCCCCCCCCC)CCN(CC(O)COC(=O)CCCCCCCCCCC)CC(O)COC(=O)CCCCCCCCCCC)CC(O)COC(=O)CCCCCCCCCCC]

A cationic lipid typically consists of a hydrophilic head group containing an amino group, a non-polar hydrophobic tail, and a linker segment that connects the head group to the tail. The structure of the head group, and the number, length and saturation of the tail have a great impact on transfection efficiency of the cationic lipid. In the present invention, a series of lipid compounds were developed by selecting organic amines with different structures, maintaining a hydroxyl-substituted three-carbon chain structure as the middle linker segment, and using saturated or unsaturated long chains with 8-18 carbon atoms as the hydrophobic tails, with their number adjusted to 2-6. These lipid compounds exhibit strong transfection efficiency and can be used for in vivo delivery of active pharmaceutical ingredients.

In some embodiments of the present invention, a preparation method for the lipid compound includes the following steps:
subjecting acyl chloride and glycidol to alcoholysis to obtain glycidyl ester, and then enabling the glycidyl ester to undergo a reaction with the organic amine to obtain the lipid compound.

In some embodiments of the present invention, a molar ratio of the acyl chloride to the glycidol is 1:1.2-1.5.

In some embodiments of the present invention, the alcoholysis is performed in the presence of an organic alkali, and the organic alkali is triethylamine.

In some embodiments of the present invention, a molar ratio of the organic alkali to the acyl chloride is 1:1-1.2.

In some embodiments of the present invention, the alcoholysis is performed at a temperature of 10-30° C.

In some embodiments of the present invention, the alcoholysis is performed for 12-36 h.

In some embodiments of the present invention, the reaction is carried out at a temperature of 80-100° C.

In some embodiments of the present invention, the reaction is carried out for 2-3 d.

In a second aspect of the present invention, a composition is provided, where the composition includes the lipid compound or a pharmaceutically acceptable salt thereof.

In some embodiments of the present invention, the composition further includes other lipid compounds.

In some embodiments of the present invention, the other lipid compounds include at least one of cholesterol, a phospholipid, and a polymer-conjugated lipid.

In some embodiments of the present invention, the phospholipid includes at least one of yolk lecithin, hydrogenated yolk lecithin, soy lecithin, hydrogenated soy lecithin, sphingomyelin, phosphatidyl ethanolamine, dimyristoyl phosphatidylcholine, dimyristoyl phosphatidylglycerol, dipalmitoyl phosphatidylcholine, distearoyl phosphatidylcholine (DSPC), dioleyl phosphatidylcholine, and dilauryl phosphatidylcholine, preferably DSPC.

In some embodiments of the present invention, the polymer-conjugated lipid includes at least one of polyethylene glycol (PEG) modified phosphatidyl ethanolamine, PEG modified phosphatidic acid, PEG modified ceramide, PEG modified dialkylamine, PEG modified diacylglycerol, and PEG modified dialkylglycerol, preferably PEG modified phosphatidyl ethanolamine.

In some embodiments of the present invention, a molar ratio of the lipid compound or the pharmaceutically acceptable salt thereof to the cholesterol is 1:0.01-9, for example, 1:0.1-9, 1:1-9, 1:1-5, or 1:1-2.

In some embodiments of the present invention, a molar ratio of the lipid compound or
the pharmaceutically acceptable salt thereof to the phospholipid is 0.5-100:1, for example, 1-100:1, 1-10:1, 1-5:1, 2-5:1, 3-5:1, or 3-4:1.

In some embodiments of the present invention, a molar ratio of the lipid compound or the pharmaceutically acceptable salt thereof to the polymer-conjugated lipid is 0.1-100:1, for example, 1-100:1, 1-50:1, 5-50:1, 10-50:1, 10-20:1, or 15-20:1.

In some embodiments of the present invention, when the other lipid compounds include the cholesterol, the phospholipid and the polymer-conjugated lipid, a molar ratio of the lipid compound or the pharmaceutically acceptable salt thereof, the cholesterol, the phospholipid and the polymer-conjugated lipid is 10-100:1-90:1-90:1-90, for example, 10-50:20-80:1-20:1-10, 20-50:30-80:1-20:1-10, 30-50:40-80:1-20:1-10, 30-40:40-70:1-20:1-10, 30-40:40-60:5-20:1-10, 30-40:40-60:5-15:1-10, or 30-40:40-60:5-15:1-5.

In some embodiments of the present invention, the composition is a lipid nanoparticle or a liposome. The lipid nanoparticle or the liposome of the present invention can be used for preparing a cell transfection reagent and has high transfection efficiency.

In some embodiments of the present invention, the composition further includes an active pharmaceutical ingredient.

In some embodiments of the present invention, a molar ratio of the lipid compound or the pharmaceutically acceptable salt thereof to the active pharmaceutical ingredient is 1-100:1.

In some embodiments of the present invention, the active pharmaceutical ingredient includes at least one of a nucleic acid molecule, a polypeptide, a protein, and a small molecule compound.

In some embodiments of the present invention, the nucleic acid molecule includes at least one of an siRNA, an mRNA, an miRNA, an antisense RNA, CRISPR guide RNAs, a replicable RNA, a cyclic dinucleotide (CDN), poly IC, CpG ODN, and a plasmid DNA, preferably an siRNA.

In some embodiments of the present invention, the protein includes at least one of a cell colony stimulating factor, interleukins, lymphotoxin, an interferon protein, and a tumor necrosis factor.

In some embodiments of the present invention, when the active pharmaceutical ingredient includes the nucleic acid molecule, a nitrogen-phosphorus ratio (N/P ratio) of the lipid compound or the pharmaceutically acceptable salt thereof to the nucleic acid molecule is 1-50:1, preferably 1-40:1, more preferably 4-32:1.

Specifically, the composition of the present invention can carry the nucleic acid molecule to penetrate through a cell membrane and can therefore be used as a transfection agent, and especially when transfected with an siRNA, can effectively inhibit the expression of a target gene.

In some embodiments of the present invention, a preparation method for the composition loaded with the active pharmaceutical ingredient includes the following steps:
mixing an ethanol solution of the lipid compound with a buffer (pH-4-6) containing the active pharmaceutical ingredient; when other lipid compounds exist, adding ethanol solutions of the other lipid compounds in the mixing process; and performing incubation at room temperature for 15-60 min and dialysis in water to obtain the composition loaded with the active pharmaceutical ingredient.

In a third aspect of the present invention, use of the lipid compound or the pharmaceutically acceptable salt thereof, or the composition in preparation of nucleic acid drugs, genetic vaccines, polypeptide or protein drugs, and small molecule drugs is provided.

The nucleic acid drugs of the present invention are drugs for treating related diseases caused by genetic abnormalities, and the diseases include monogenic diseases, such as methemoglobinemia and sickle cell anemia; polygenic diseases, such as neoplasms, cardiovascular diseases, metabolic diseases, neurological and psychiatric diseases, and immune diseases; and acquired genetic diseases, such as acquired immune deficiency syndrome.

The lipid compound or the composition according to the embodiments of the present invention at least has the following beneficial effects.

In the prior art, a hydrophobic end of a lipid is composed of an alkane or an olefin with a long carbon chain, which is difficult to degrade by an enzyme and difficult to metabolize in vivo. The lipid compound of the present invention introduces a biodegradable ester bond at the hydrophobic end, which can be degraded by esterase in vivo, such that the lipid compound is easy to metabolize and clear. In addition, the protonable lipid compound prepared by the present invention can be ionized into cations under acidic conditions, can be bonded to the negatively charged active pharmaceutical ingredient through charge interaction, and can also be further combined with other lipid compounds, such as DSPC, cholesterol, DSPE-PEG, etc., to form a lipid nanoparticle so as to effectively deliver the active pharmaceutical ingredient to cells or tissues. For example, the siRNA can be transfected into cells to specifically knock out a target gene and inhibit the expression of the target gene. Data in the embodiments also indicate that the lipid compound prepared by the present invention has high transfection efficiency. In addition, the present invention has the advantages of easily available raw materials, a simple reaction and a high yield.

In the present application, the term “pharmaceutically acceptable salt” includes conventional salts formed with pharmaceutically acceptable inorganic acids or organic acids, or inorganic alkalies or organic alkalies.

The “composition” includes a product containing an effective amount of the compound of the present invention and any products that are directly or indirectly produced from combinations of the compounds of the present application.

### BRIEF DESCRIPTION OF THE DRAWINGS

The present invention is further described below in conjunction with the accompanying drawings and embodiments.

FIG. 1 is a Fourier infrared scanning image of C12 in the present invention.

FIG. 2 shows a nuclear magnetic resonance hydrogen spectrum of the C12 in the present invention.

FIG. 3 shows a nuclear magnetic resonance hydrogen spectrum of C16 in the present invention.

FIG. 4 shows a nuclear magnetic resonance hydrogen spectrum of A1-C12 in the present invention.

FIG. 5 shows a nuclear magnetic resonance hydrogen spectrum of A2-C12 in the present invention.

FIG. 6 shows a nuclear magnetic resonance hydrogen spectrum of A2-C16 in the present invention.

FIG. 7 shows a nuclear magnetic resonance hydrogen spectrum of A2-C18U in the present invention.

FIG. 8 shows a nuclear magnetic resonance hydrogen spectrum of A13-C16 in the present invention.

FIG. 9 shows a nuclear magnetic resonance hydrogen spectrum of A13-C18U in the present invention.

FIG. 10 shows a nuclear magnetic resonance hydrogen spectrum of A12-C12 in the present invention.

FIG. 11 shows an expression amount of firefly luciferase after lipid nanoparticles constructed by A1-C8, A1-C10, A1-C12, A1-C16 and A1-C18U of the present invention are coated with a firefly luciferase small interfering RNA (luciferase siRNA, siLuc), where 4:1, 8:1 and 16:1 indicate nitrogen-phosphorus ratios of lipid compounds to the siRNA, namely, molar ratios of protonable amino on the lipid compounds to a phosphate group on the nucleic acid.

FIG. 12 shows an expression amount of firefly luciferase after lipid nanoparticles constructed by A2-C8, A2-C10, A2-C12, A2-C14, A2-C16 and A2-C18U of the present invention are coated with siLuc, where 4:1, 8:1 and 16:1 indicate nitrogen-phosphorus ratios of lipid compounds to the siRNA, namely, molar ratios of protonable amino on the lipid compounds to a phosphate group on the nucleic acid.

FIG. 13 is a heat map of transfection efficiency of lipid nanoparticles constructed by different lipid compounds of the present invention at different nitrogen-phosphorus ratios, where the nitrogen-phosphorus ratios are 4:1, 8:1 and 16:1, respectively, and a value of each unit in the heat map represents the transfection efficiency.

FIG. 14 shows an expression amount of firefly luciferase after lipid nanoparticles constructed by A5-C12, A6-C12, A7-C12, A8-C12, A9-C12, A10-C12, A11-C12, A12-C12 and A13-C12 of the present invention are coated with siLuc, where 4:1, 8:1, 16:1 and 32:1 indicate nitrogen-phosphorus ratios of lipid compounds to the siRNA, namely, molar ratios of protonable amino on the lipid compounds to a phosphate group on the nucleic acid.

FIG. 15 is a heat map of transfection efficiency of lipid nanoparticles constructed by different lipid compounds of the present invention at different nitrogen-phosphorus ratios, where the nitrogen-phosphorus ratios are 4:1, 8:1, 16:1 and 32:1, respectively, and a value of each unit in the heat map represents the transfection efficiency.

FIG. 16 shows fluorescence microscope images of lipid nanoparticles prepared by A12-C12, A13-C16 and A7-C12 of the present invention after transfection with a plasmid DNA of a green fluorescent protein for 48 h, where polyethylenimine (PEI) is a commercial transfection reagent.

FIG. 17 is a histogram of efficiency of protonable lipid compounds transfected with a plasmid DNA of firefly luciferase at different nitrogen-phosphorus ratios 48 h after transfection.

### DESCRIPTION OF THE EMBODIMENTS

Concepts of the present invention and resulting technical effects are clearly and completely described below in combination with embodiments to make purposes, features and effects of the present invention fully understood. Obviously, the described embodiments are only a part of the embodiments of the present invention, rather than all of the embodiments. On the basis of the embodiments of the present invention, all other embodiments obtained by persons skilled in the art without exerting creative efforts should fall within the scope of protection of the present invention.

### EMBODIMENTS

### Example 1 Synthesis of an Intermediate C12

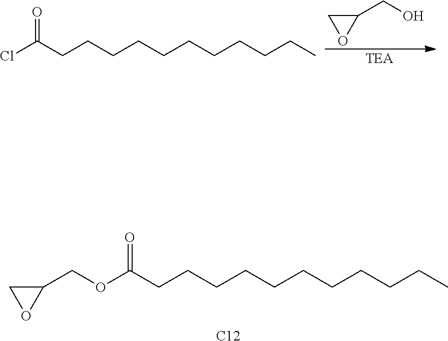

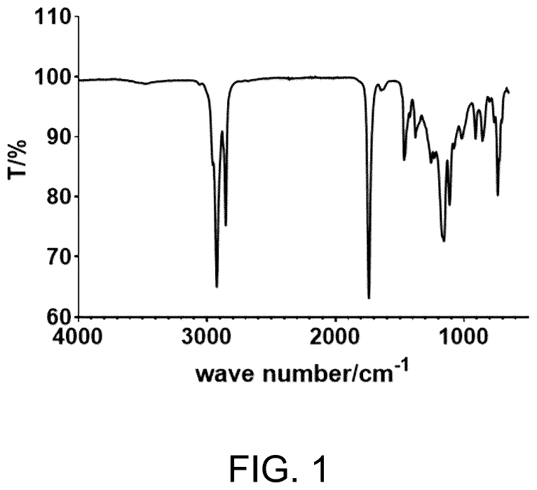

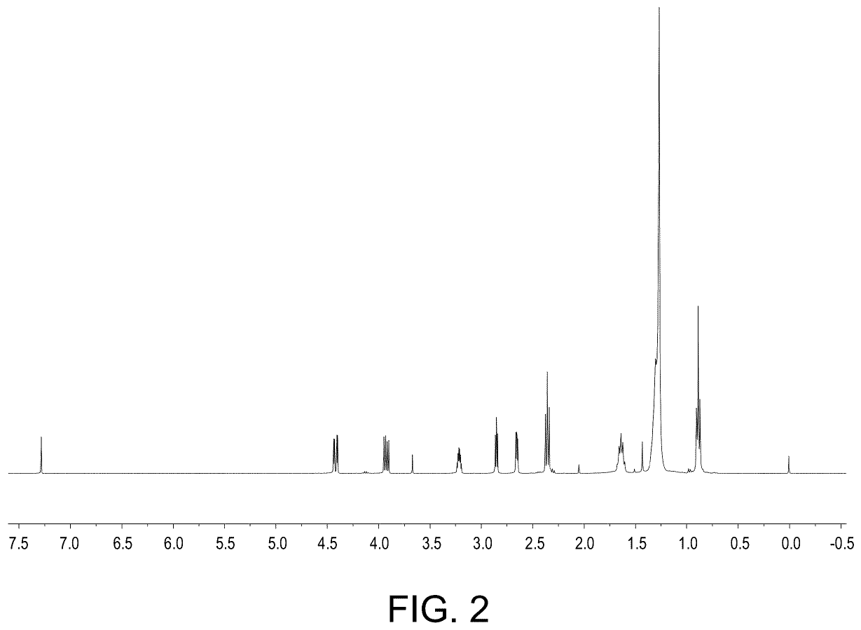

Glycidol and lauroyl chloride reacted at a molar ratio of 1.2:1.0. Specific operations are as follows. The glycidol was dissolved in anhydrous dichloromethane and placed in a 25 mL round-bottomed flask with a stopper, a catalytic amount of triethylamine (TEA) was added and mixed, and then, a mixed solution was sealed and pre-cooled in an ice bath for 30 min. Under magnetic stirring, a dichloromethane solution of the lauroyl chloride was slowly added dropwise to the mixed solution of the glycidol and the TEA by a constant-pressure dropping funnel, a dropping rate was controlled, and after the dropping was completed, a reaction was carried out at room temperature overnight. Washing was performed with a saturated sodium bicarbonate solution for two times and with a saturated sodium chloride solution for 1 time. An organic phase was taken, concentrated, dried with anhydrous magnesium sulfate for 30 min, and then purified by a 200- to 300-mesh silica gel column to obtain a product C12. The obtained product has an infrared characterization structure as shown in FIG. 1 and was characterized by nuclear magnetic resonance as shown in FIG. 2.

### Example 2 Synthesis of an Intermediate C16

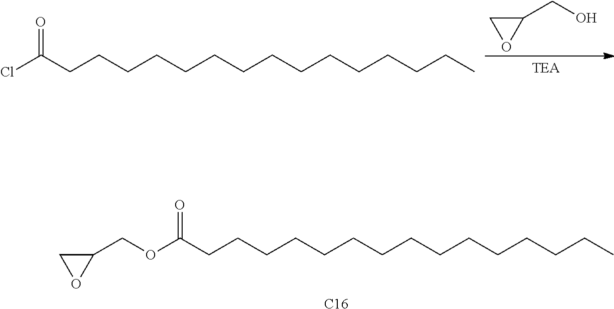

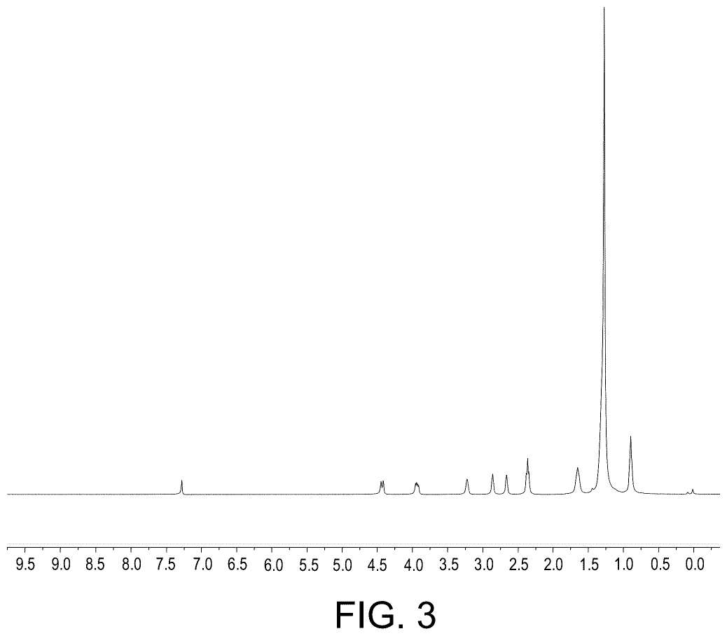

Glycidol and palmitoyl chloride reacted at a molar ratio of 1.2:1.0. Specific operations are as follows. The glycidol was dissolved in anhydrous dichloromethane and placed in a 25 mL round-bottomed flask with a stopper, a catalytic amount of TEA was added and mixed, and then, a mixed solution was sealed and pre-cooled in an ice bath for 30 min. Under magnetic stirring, a dichloromethane solution of the palmitoyl chloride was slowly added dropwise to the mixed solution of the glycidol and the TEA by a constant-pressure dropping funnel, a dropping rate was controlled, and after the dropping was completed, a reaction was carried out at room temperature overnight. Washing was performed with a saturated sodium bicarbonate solution for two times and with a saturated sodium chloride solution for 1 time. An organic phase was taken, concentrated, dried with anhydrous magnesium sulfate for 30 min, and then purified by a 200- to 300-mesh silica gel column to obtain a product C16. The obtained product was characterized by nuclear magnetic resonance as shown in FIG. 3.

### Example 3 Synthesis of an Intermediate C18U

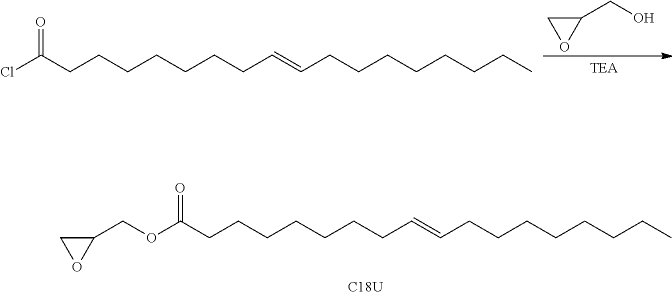

Glycidol and oleoyl chloride reacted at a molar ratio of 1.2:1.0. Specific operations are as follows. The glycidol was dissolved in anhydrous dichloromethane and placed in a 25 mL round-bottomed flask with a stopper, a catalytic amount of TEA was added and mixed, and then, a mixed solution was sealed and pre-cooled in an ice bath for 30 min. Under magnetic stirring, a dichloromethane solution of the oleoyl chloride was slowly added dropwise to the mixed solution of the glycidol and the TEA by a constant-pressure dropping funnel, a dropping rate was controlled, and after the dropping was completed, a reaction was carried out at room temperature overnight.

Washing was performed with a saturated sodium bicarbonate solution for two times and with a saturated sodium chloride solution for 1 time. An organic phase was taken, concentrated, dried with anhydrous magnesium sulfate for 30 min, and then purified by a 200- to 300-mesh silica gel column to obtain a product C18U.

### Example 4 Synthesis of a Lipid Compound A1-C12

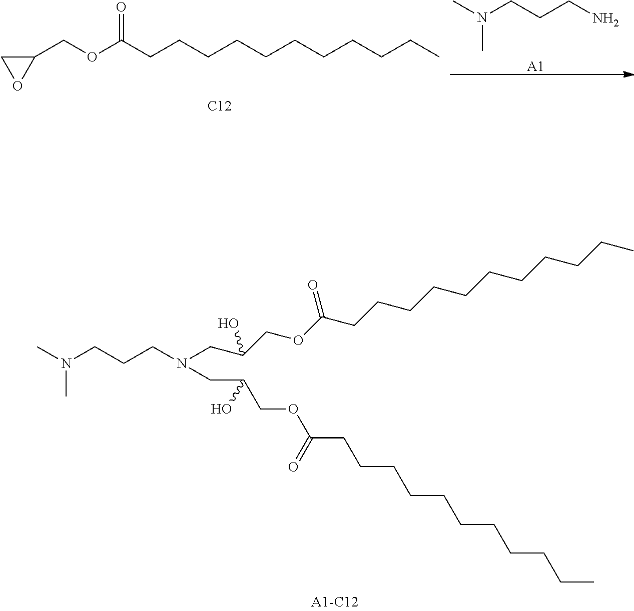

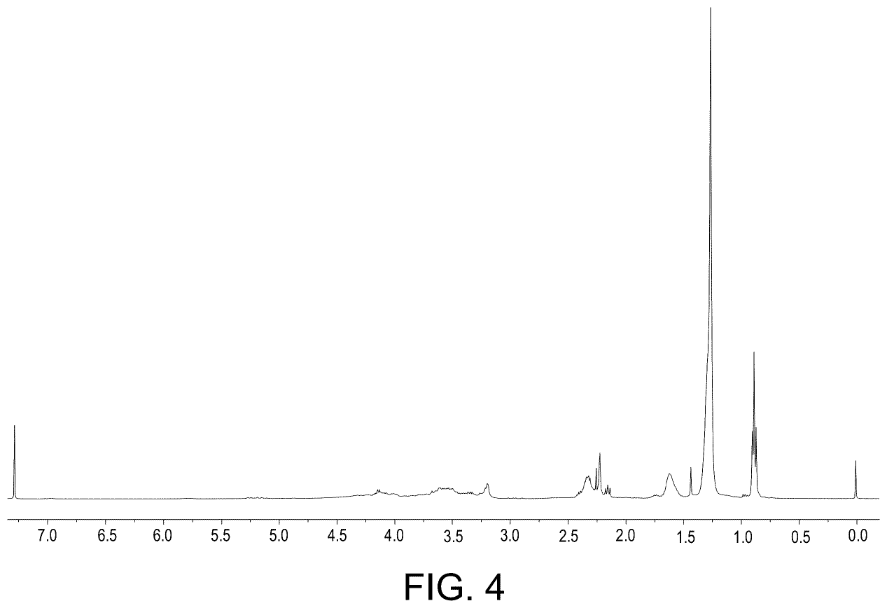

The intermediate C12 (glycidyl laurate) and a compound A1 reacted at a molar ratio of 2.4:1. Specific operations are as follows. Certain amounts of the intermediate C12 and the compound A1 were weighed, placed in a 2 mL glass flask, and then placed in a magnetic stirrer to carry out a reaction at 90° C. for 72 h to obtain a lipid compound A1-C12. Nuclear magnetic resonance is shown in FIG. 4. ¹H NMR (400 MHz, CDCl₃) δ 0.85-0.88 (t, 6H), 1.24-1.26 (m, 32H), 1.60-1.66 (m, 6H), 2.14-2.19 (t, 6H), 2.31-2.43 (m, 12H), 4.08-4.19 (m, 8H).

### Example 5 Synthesis of a Lipid Compound A2-C12

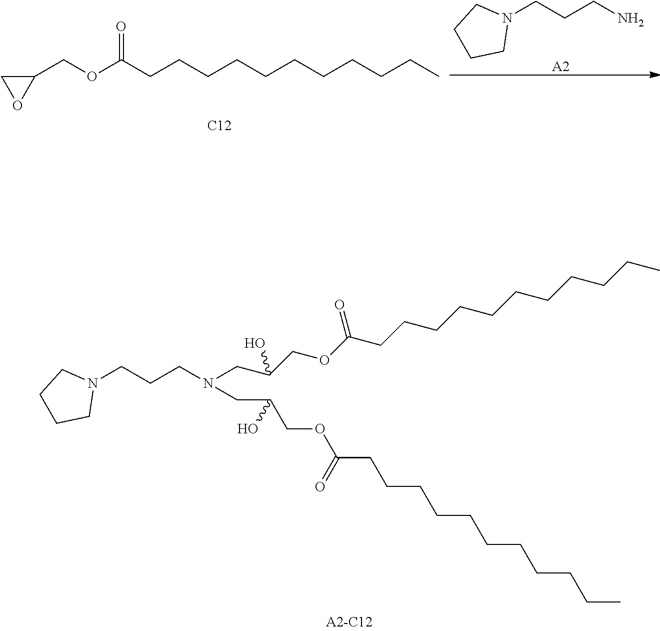

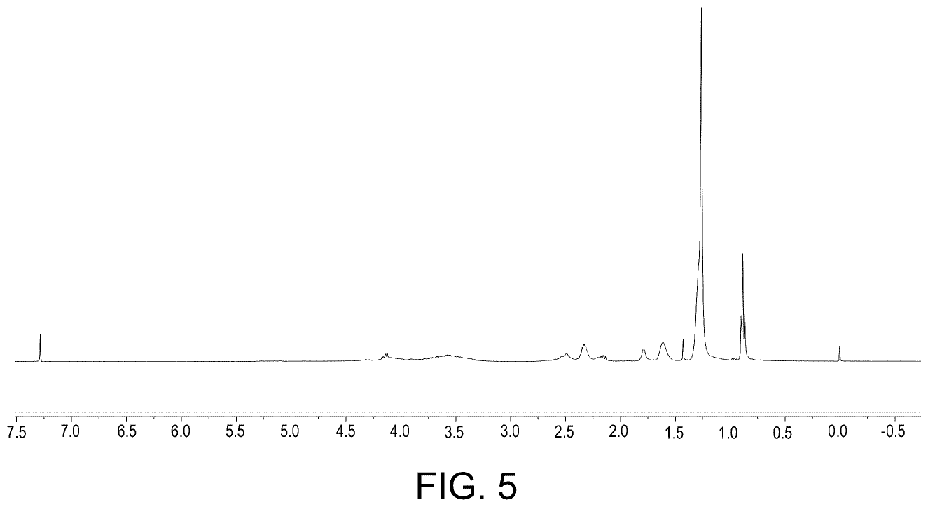

The intermediate C12 (glycidyl laurate) and a compound A2 reacted at a molar ratio of 2.4:1. Specific operations are as follows. Certain amounts of the intermediate C12 and the compound A2 were weighed, placed in a 2 mL glass flask, and then placed in a magnetic stirrer to carry out a reaction at 90° C. for 72 h to obtain a lipid compound A2-C12. Nuclear magnetic resonance is shown in FIG. 5. ¹H NMR (400 MHZ, CDCl₃): δ 0.86-0.90 (t, 6H), 1.21-1.30 (m, 32H), 1.56-1.68 (m, 10H), 2.03-2.33 (m, 16H), 4.08-4.14 (m, 8H).

### Example 6 Synthesis of a Lipid Compound A2-C16

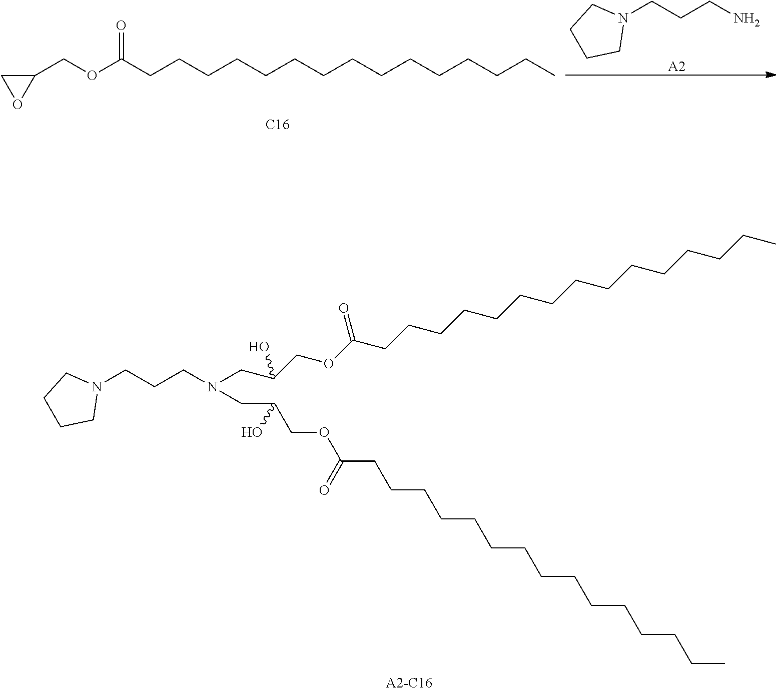

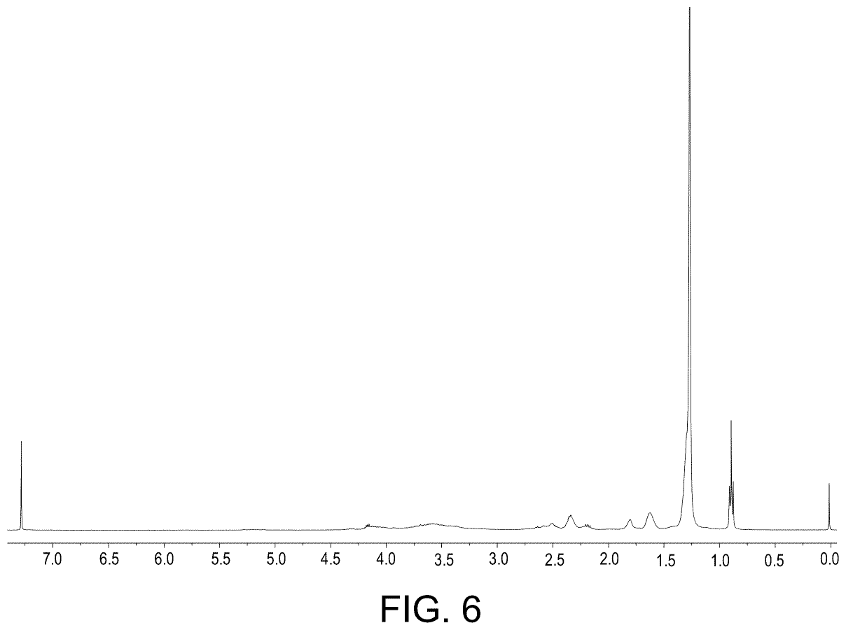

The intermediate C16 (glycidyl palmitate) and a compound A2 reacted at a molar ratio of 2.4:1. Specific operations are as follows. Certain amounts of the intermediate C16 and the compound A2 were weighed, placed in a 2 mL glass flask, and then placed in a magnetic stirrer to carry out a reaction at 90° C. for 72 h to obtain a lipid compound A2-C16. Nuclear magnetic resonance is shown in FIG. 6. ¹H NMR (400 MHZ, CDCl₃): δ 0.88-0.91 (t, 6H), 1.27 (m, 48H), 1.56-1.68 (m, 10H), 2.32-2.52 (m, 16H), 4.08-4.14 (m, 8H).

### Example 7 Synthesis of a Lipid Compound A2-C18U

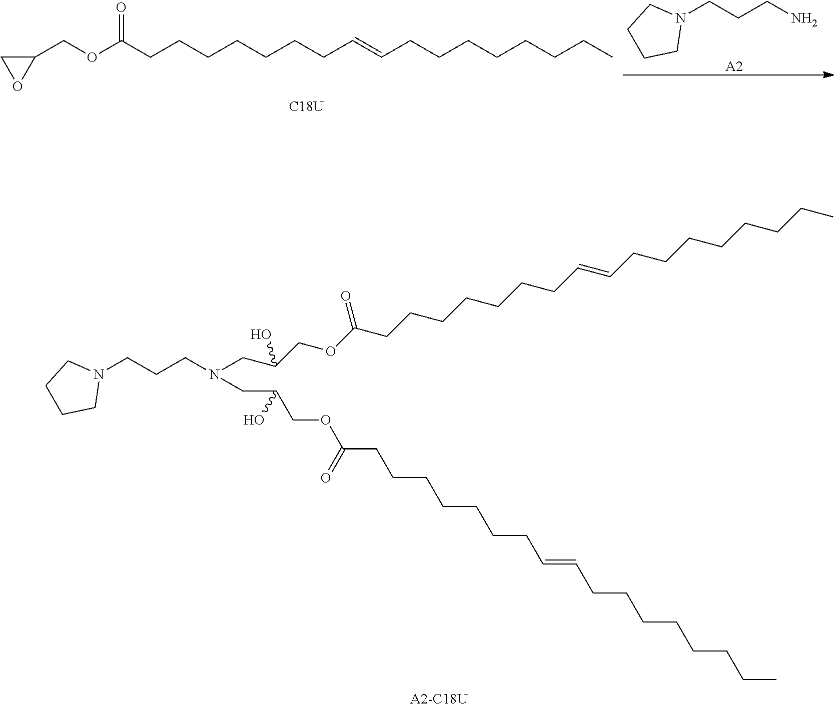

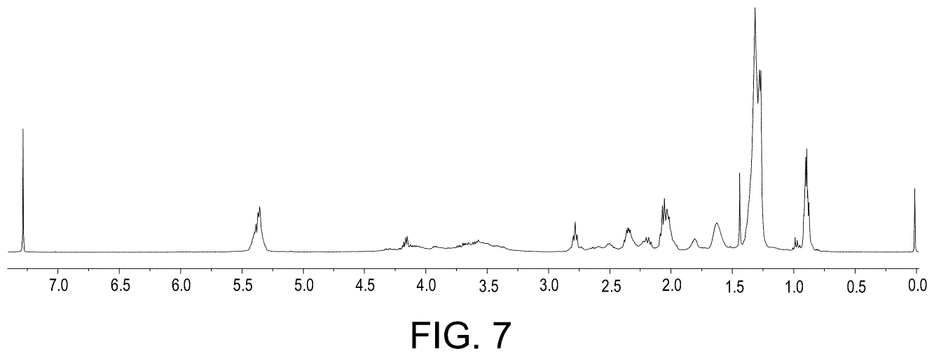

The intermediate C18U (glycidyl palmitate) and a compound A2 reacted at a molar ratio of 2.4:1. Specific operations are as follows. Certain amounts of the intermediate C18U and the compound A2 were weighed, placed in a 2 mL glass flask, and then placed in a magnetic stirrer to carry out a reaction at 90° C. for 72 h to obtain a lipid compound A2-C18U. Nuclear magnetic resonance is shown in FIG. 7. ¹H NMR (400 MHZ, CDCl₃): δ 0.85-0.92 (t, 6H), 1.27-1.40 (m, 40H), 1.56-1.68 (m, 10H), 2.02-2.14 (m, 8H), 2.20-2.24 (m, 16H), 4.08-4.14 (m, 8H), 5.37-5.40 (m, 4H).

### Example 8 Synthesis of a Lipid Compound A13-C16

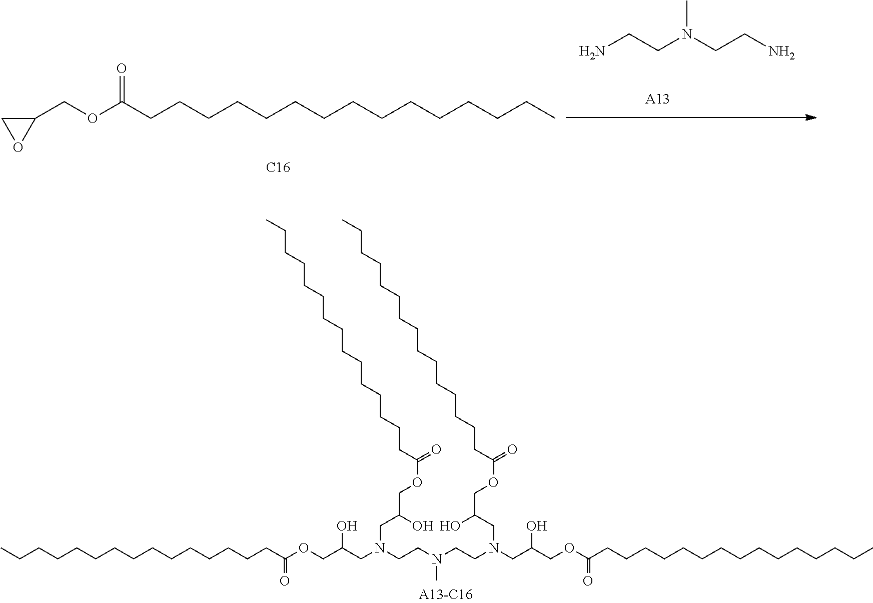

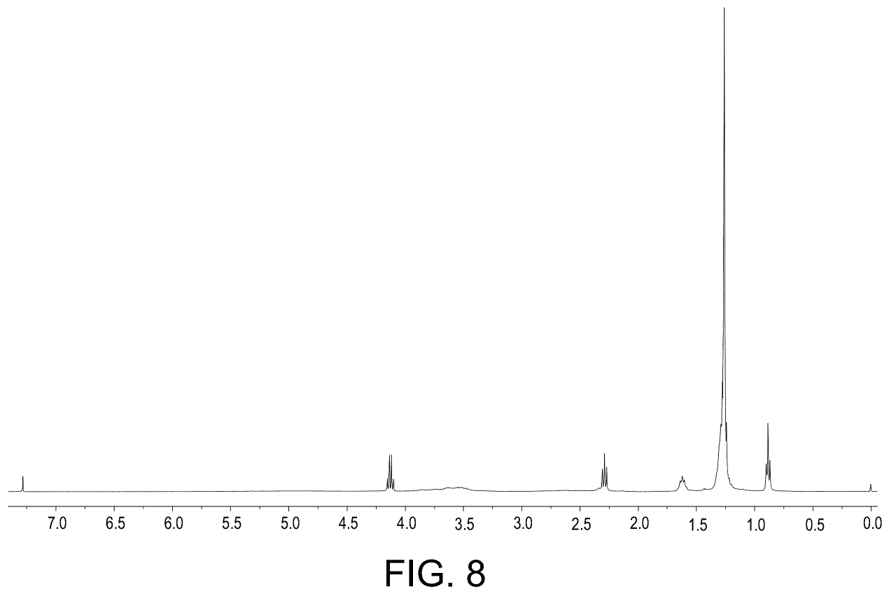

The intermediate C16 (glycidyl palmitate) and a compound A13 reacted at a molar ratio of 5:1. Specific operations are as follows. Certain amounts of the intermediate C16 and the compound A13 were weighed, placed in a 2 mL glass flask, and then placed in a magnetic stirrer to carry out a reaction at 90° C. for 72 h to obtain a lipid compound A13-C16. Nuclear magnetic resonance is shown in FIG. 8. ¹H NMR (400 MHZ, CDCl₃): δ 0.87-0.98 (t, 12H), 1.20-1.29 (m, 96H), 1.58-1.66 (t, 8H), 2.14-2.28 (brs, 3H), 2.32-2.47 (m, 24H), 4.08-4.33 (m, 12H).

### Example 9 Synthesis of a Lipid Compound A13-C18U

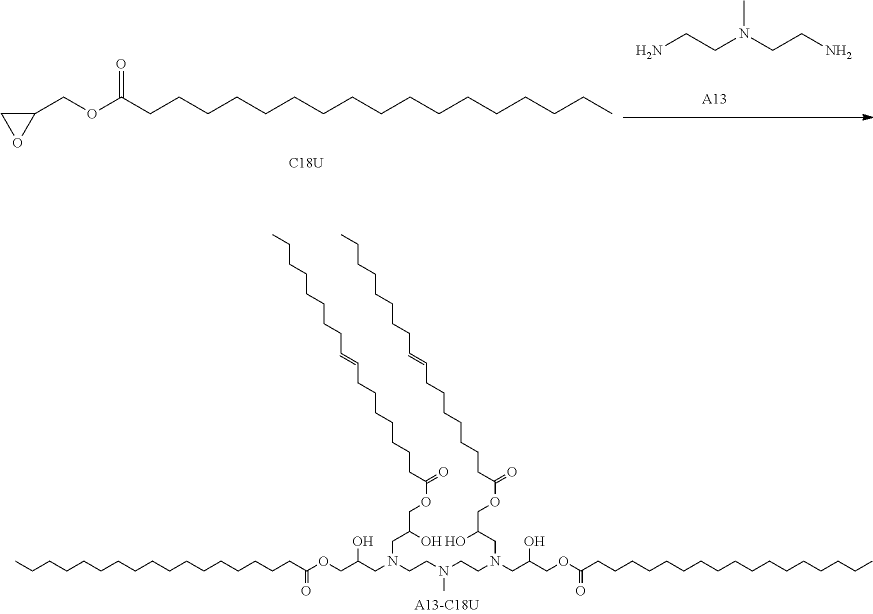

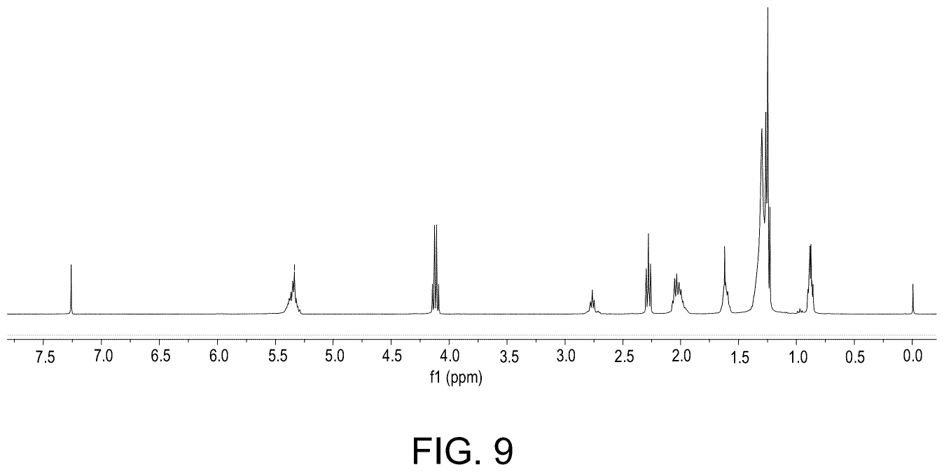

The intermediate C18U and a compound A13 reacted at a molar ratio of 5:1. Specific operations are as follows. Certain amounts of the intermediate C18U and the compound A13 were weighed, placed in a 2 mL glass flask, and then placed in a magnetic stirrer to carry out a reaction at 90° C. for 72 h to obtain a lipid compound A13-C18U. Nuclear magnetic resonance is shown in FIG. 9. ¹H NMR (400 MHZ, CDCl₃): δ 0.86-0.90 (t, 12H), 1.13-1.42 (m, 88H), 1.58-1.66 (m, 8H), 1.96-2.30 (m, 19H), 2.40-2.76 (m, 16H), 4.09-4.14 (m, 12H), 5.35 (m, 12H).

### Example 10 Synthesis of a Lipid Compound A12-C12

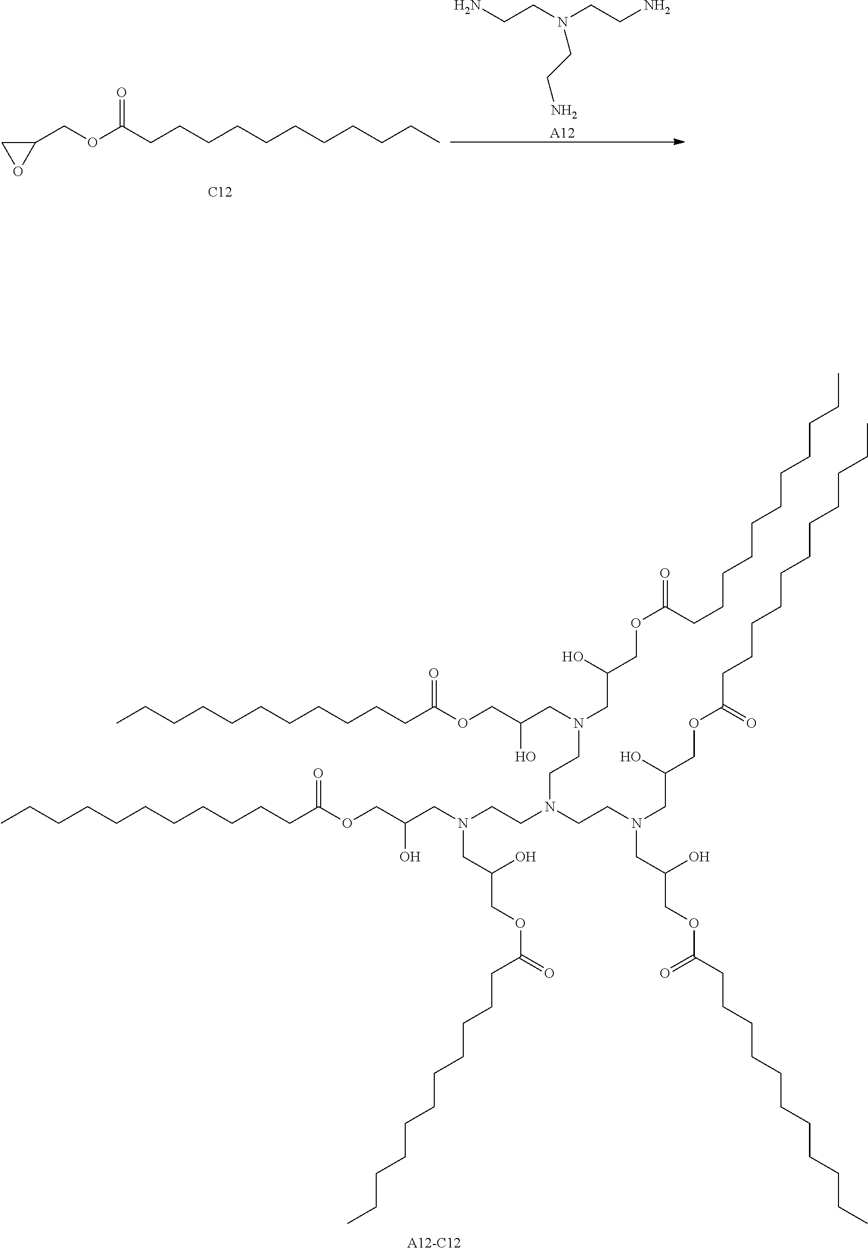

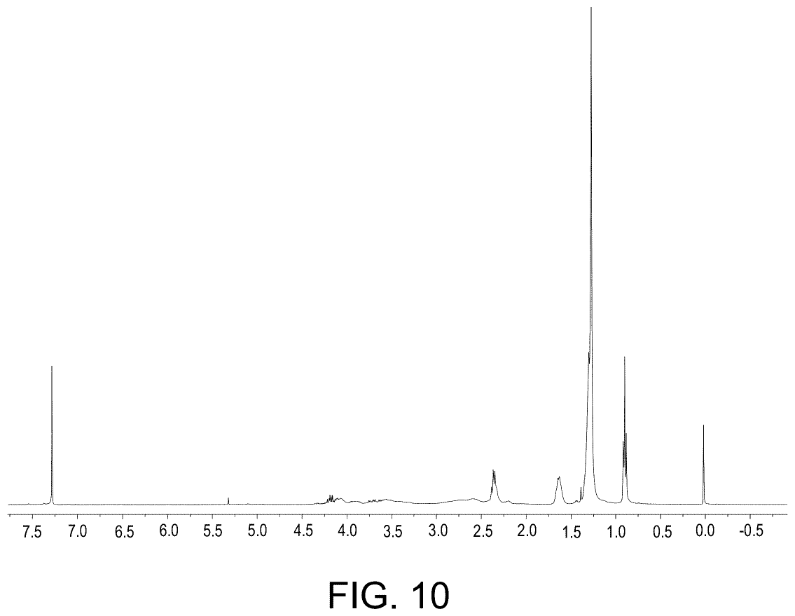

The intermediate C12 and a compound A12 reacted at a molar ratio. Specific operations are as follows. Certain amounts of the intermediate C12 and the compound A12 were weighed, placed in a 2 mL glass flask, and then placed in a magnetic stirrer to carry out a reaction at 90° C. for 72 h to obtain a lipid compound A12-C12. Nuclear magnetic resonance is shown in FIG. 10. ¹H NMR (400 MHZ, CDCl₃): δ 0.88-0.92 (t, 18H), 1.28-1.35 (m, 104H), 1.63-1.65 (m, 12H), 2.33-2.39 (m, 30H), 4.08-4.33 (m, 24H).

A reaction mechanism of the lipid compound of the present invention is as follows. A ternary epoxy compound, due to large tension of ring, extremely low chemical bond strength and high system energy, easily undergoes a ring-opening reaction with amino having strong nucleophilicity, so as to obtain the lipid compound of the present invention. The reaction mechanism is highly mature, and a reaction process is also well known in the field. Therefore, a specific type and a reaction degree of the compound generated based on the reaction mechanism are completely predictable. Reaction conditions and structural characterization of some compounds synthesized in the present invention are described above, synthesis of other compounds of the present invention is the same as that of the above compounds, and structural formulas and structural characterization data of the other compounds are not described in detail herein.

### Example 11

Lipid compounds A1-C8, A1-C10, A1-C12, A1-C14, A1-C16 and A1-C18U were used as a drug carrier to transfect siLuc into a melanoma (B16F10-Luc) cell line capable of stably expressing firefly luciferase (Luc), respectively. Specific steps are as follows.

B16F10-Luc cells were inoculated into a 96-well cell culture plate. On the next day, transfection was performed when the cells grew to about 80%.

Experimental group: The prepared protonable lipid compounds A1-C8, A1-C10, A1-C12, A1-C14, A1-C16 and A1-C18U as well as distearoyl phosphatidylcholine (DSPC), cholesterol and distearoyl phosphatidylacetamide-polyethylene glycol (DSPE-PEG) were dissolved in anhydrous ethanol to prepare respective mother solutions, respectively. The mother solutions were stored in a refrigerator at −20° C., diluted as needed during use, and then mixed at a molar ratio of 38:10:50:2 (lipid compounds:DSPC:cholesterol:DSPE-PEG). The siLuc was dissolved in a citrate buffer solution (pH-4), where a volume of the citrate buffer solution was two times the volume of an ethanol-lipid mixture obtained above. Finally, the citrate buffer solution containing siLuc (pH=4) and the ethanol-lipid mixture were quickly and fully mixed and then shaken and incubated at room temperature for 30 min for self-assembly to form lipid nanoparticles. The assembled lipid nanoparticles were added to the 96-well cell culture plate containing the B16F10-Luc for transfection, respectively. Before the transfection, a culture solution in the culture plate was sucked out, and 80 μL of a new culture medium was added, where an added amount of the siRNA was 50 ng/well. Nitrogen-phosphorus ratios (N/P ratio) of the protonable lipid compounds to the siRNA were 4:1, 8:1 and 16:1, respectively.

Positive control group: The siLuc was transfected by Lipo2000, a commercial transfection reagent. Transfection was performed according to an operating instruction of the Lipo2000. 50 ng of the siLuc was added to 5 μL of Opti-MEM, and 0.3 μL of the Lipo2000 was added to another 50 μL of Opti-MEM. Finally, an siRNA Opti-MEM solution was added to a Lipo2000 Opti-MEM solution, evenly mixed, incubated at room temperature for 15 min, and then added to a 96-well cell culture plate. Before the transfection, a culture solution in the culture plate was sucked out, and 80 μL of a new culture medium was added, where an added amount of the siRNA was 50 ng/well.

Negative control group: Only the B16F10-Luc cells were used without transfection.

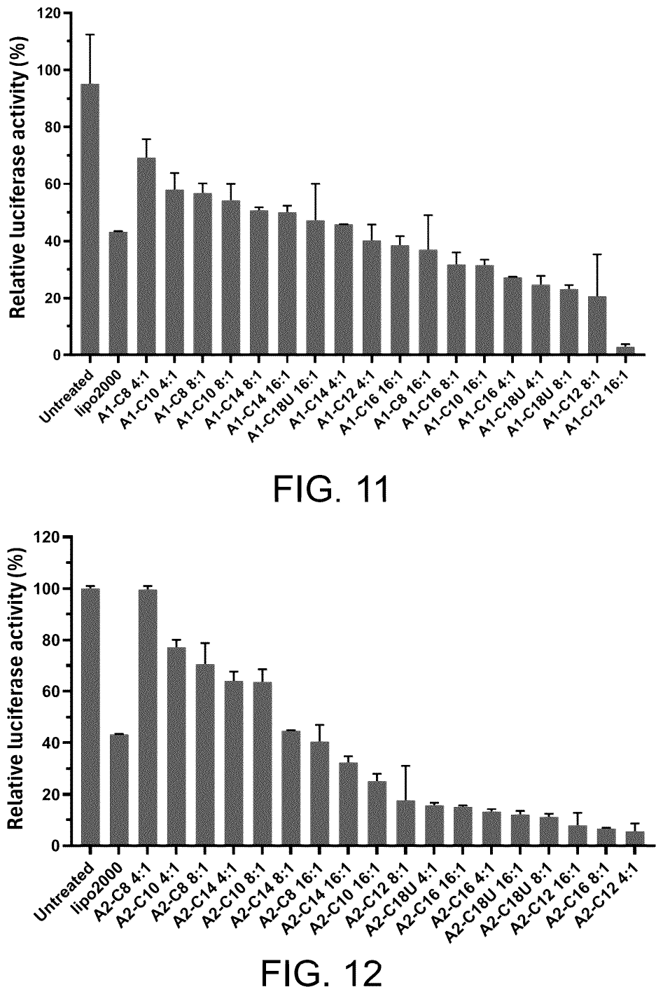

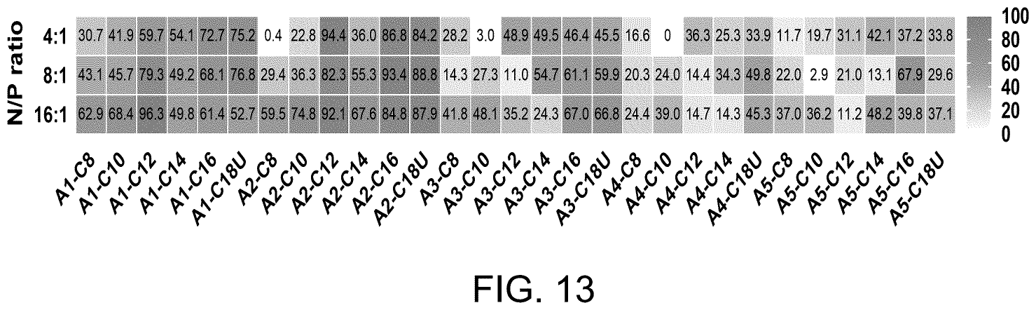

After the transfection was performed for 24 h, the cells were lysed and subjected to centrifugation to remove cell debris and contents, a supernatant was taken, a firefly luciferase substrate was added, and an expression amount of firefly luciferase was determined so as to compare the siLuc transfection efficiency of the synthesized lipid compounds. Test results are shown in FIG. 11 and FIG. 13. Most of the synthesized lipid compounds have relatively high transfection efficiency. The transfection efficiency of the A1-C12 can reach about 95%.

### Example 12

Lipid compounds A2-C8, A2-C10, A2-C12, A2-C14, A2-C16 and A2-C18U were used as a genetic vector material to transfect siLuc into a B16F10-Luc cell line, respectively. Specific steps are as follows.

B16F10-Luc cells were inoculated into a 96-well cell culture plate. On the next day, transfection was performed when the cells grew to about 80%.

Experimental group: The prepared lipid compounds A2-C8, A2-C10, A2-C12, A2-C14, A2-C16 and A2-C18U as well as DSPC, cholesterol and DSPE-PEG were dissolved in anhydrous ethanol to prepare respective mother solutions, respectively. The mother solutions were stored in a refrigerator at −20° C., diluted as needed during use, and then mixed at a molar ratio of 38:10:50:2 (lipid compounds:DSPC:cholesterol:DSPE-PEG). The siLuc was dissolved in a citrate buffer solution (pH=4), where a volume of the citrate buffer solution was two times the volume of an ethanol-lipid mixture obtained above. The citrate buffer solution containing siLuc (pH=4) and the ethanol-lipid mixture were quickly and fully mixed and then shaken and incubated at room temperature for 30 min for self-assembly to form lipid nanoparticles. Then, the assembled lipid nanoparticles were added to the 96-well cell culture plate containing the B16F10-Luc cells for transfection, respectively. Before the transfection, a culture solution in the culture plate was changed, and 80 μL of a new culture medium was added, where an added amount of the siRNA was 50 ng/well. Nitrogen-phosphorus ratios of the lipid compounds to the siRNA were 4:1, 8:1 and 16:1, respectively.

Positive control group: The siLuc was transfected by a Lipo2000 transfection reagent. Transfection was performed according to an operating instruction of the Lipo2000. 50 ng of the siLuc was added to 5 μL of Opti-MEM, and 0.3 mL of the Lipo2000 was added to another 50 μL of Opti-MEM. Finally, an siRNA Opti-MEM solution was added to a Lipo2000 Opti-MEM solution, evenly mixed, incubated at room temperature for 15 min, and then added to a 96-well cell culture plate. Before the transfection, a culture solution in the culture plate was sucked out, and 80 μL of a new culture medium was added, where an added amount of the siRNA was 50 ng/well.

Negative control group: Only the B16F10-Luc cells were used without transfection.

After the transfection was performed for 24 h, the cells were lysed and subjected to centrifugation to remove cell debris and contents, a supernatant was taken, a firefly luciferase substrate was added, and an expression amount of firefly luciferase was determined so as to compare the siLuc transfection efficiency of the synthesized lipid compounds. Test results are shown in FIG. 12 and FIG. 13. Most of the synthesized lipid compounds have relatively strong transfection efficiency. The transfection efficiency of the A2-C12 and the A2-C16 can reach about 95%.

### Example 13

Lipid compounds A5-C12, A5-C16, A6-C12, A7-C12, A7-C16, A8-C12, A8-C16, A9-C12, A9-C16, A10-C16, A10-C12, A11-C16, A11-C12, A12-C12, A12-C16, A13-C12 and A13-C16 were used as a genetic vector material to transfect siLuc into a B16F10-Luc cell line, respectively. Specific steps are as follows.

B16F10-Luc cells were inoculated into a 96-well cell culture plate. On the next day, transfection was performed when the cells grew to about 80%.

Experimental group: The prepared protonable lipid compounds A5-C12, A5-C16, A6-C12, A7-C12, A7-C16, A8-C12, A8-C16, A9-C12, A9-C16, A10-C16, A10-C12, A11-C16, A11-C12, A12-C12, A12-C16, A13-C12 and A13-C16 as well as DSPC, cholesterol and DSPE-PEG were dissolved in anhydrous ethanol to prepare respective mother solutions, respectively. The mother solutions were stored in a refrigerator at −20° C., diluted as needed during use, and then mixed at a molar ratio of 38:10:50:2 (lipid compounds:DSPC:cholesterol:DSPE-PEG). The siLuc was dissolved in a citrate buffer solution (pH=4), where a volume of the citrate buffer solution was two times the volume of an ethanol-lipid mixture obtained above. The citrate buffer solution containing siLuc (pH-4) and the ethanol-lipid mixture were quickly and fully mixed and then shaken and incubated at room temperature for 30 min for self-assembly to form lipid nanoparticles. Then, the assembled lipid nanoparticles were added to the culture plate containing the B16F10-Luc cells for transfection, respectively. Nitrogen-phosphorus ratios of the lipid compounds to the siRNA were 4:1, 8:1, 16:1 and 32:1, respectively.

Positive control group: The siLuc was transfected by a Lipo2000 transfection reagent. Transfection was performed according to an operating instruction of the Lipo2000. 50 ng of the siLuc was added to 5 μL of Opti-MEM, and 0.3 μL of the Lipo2000 was added to another 50 μL of Opti-MEM. Finally, an siRNA Opti-MEM solution was added to a Lipo2000 Opti-MEM solution, evenly mixed, incubated at room temperature for 15 min, and then added to a 96-well cell culture plate. Before the transfection, a culture solution in the culture plate was sucked out, and 80 μL of a new culture medium was added, where an added amount of the siRNA was 50 ng/well.

Negative control group: Only the B16F10-Luc cells were used without transfection.

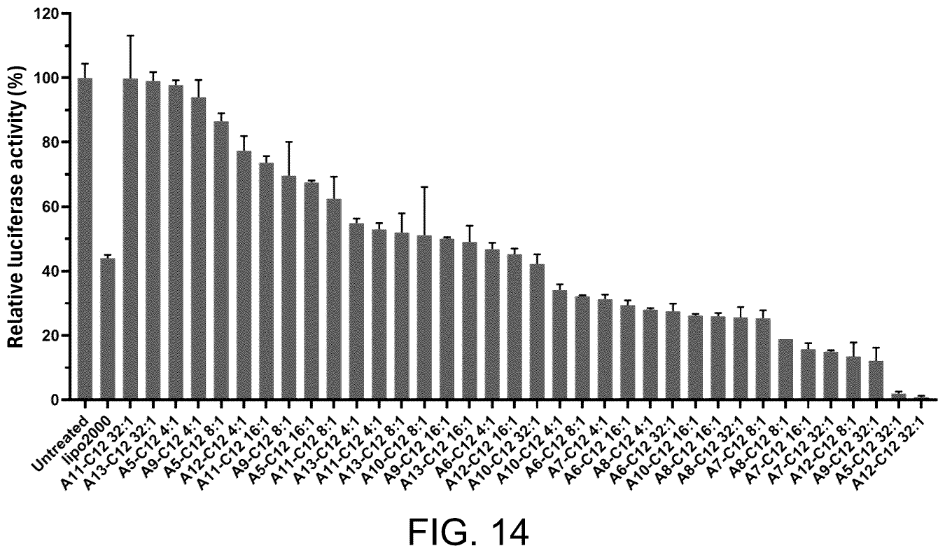

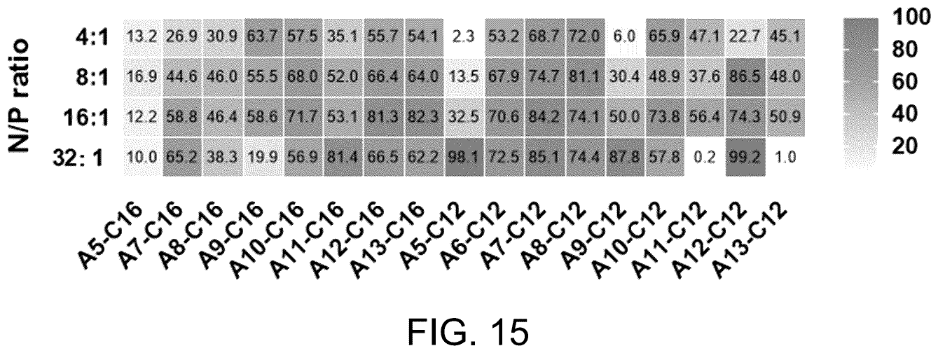

After the transfection was performed for 24 h, the cells were lysed and subjected to centrifugation to remove cell debris and contents, a supernatant was taken, a firefly luciferase substrate was added, and an expression amount of firefly luciferase was determined so as to compare the siLuc transfection efficiency of the synthesized lipid compounds. Test results are shown in FIG. 14 and FIG. 15. Most of the synthesized lipid compounds have relatively strong transfection efficiency. The transfection efficiency of the A12-C12, the A5-C12 and the A8-C12 can reach 90% or above. The A12-C12 almost completely inhibited the gene expression of the firefly luciferase.

### Example 14

Lipid compounds A1-C12, A1-C16, A1-C18U, A2-C12, A2-C16, A2-C18U, A7-C12, A13-C16, A12-C16, A8-C12 and A12-C12 were used as a genetic vector material to transfect a plasmid DNA (pDNA-GFP-Luc) of a green fluorescent protein and firefly luciferase into a 293T cell line, respectively. Specific steps are as follows.

293T cells were inoculated into a 96-well cell culture plate. On the next day, transfection was performed when the cells grew to about 80%.

Experimental group: The prepared lipid compounds A1-C12, A1-C16, A1-C18U, A2-C12, A2-C16, A2-C18U, A7-C12, A13-C16, A12-C16, A8-C12 and A12-C12 as well as DSPC, cholesterol and DSPE-PEG were dissolved in anhydrous ethanol to prepare respective mother solutions, respectively. The mother solutions were stored in a refrigerator at −20° C., diluted as needed during use, and then mixed at a molar ratio of 38:10:50:2 (lipid compounds:DSPC:cholesterol:DSPE-PEG). The plasmid DNA (pDNA-GFP-Luc) expressing the green fluorescent protein and the firefly luciferase was dissolved in a citrate buffer solution (pH=4), where a volume of the citrate buffer solution was two times the volume of an ethanol-lipid mixture obtained above. The citrate buffer solution containing plasmid DNA (pH=4) and the ethanol-lipid mixture were quickly and fully mixed and then shaken and incubated at room temperature for 30 min for self-assembly to form lipid nanoparticles. Then, the assembled lipid nanoparticles were added to the culture plate containing the 293T cells for transfection, respectively. Before the transfection, a culture solution in the culture plate was sucked out, and 80 μL of a new culture medium was added, where an added amount of the DNA was 80 ng/well. Nitrogen-phosphorus ratios of the protonable lipid compounds to the plasmid were 8:1, 16:1 and 32:1, respectively.

Positive control group: The 293T cells were transfected by a PEI commercial transfection reagent. Transfection was performed according to an instruction of the PEI transfection reagent. 80 ng of the DNA was placed in 5 μL of ddH2O and evenly mixed, and 0.1 μL of the PEI was placed in 5 μL of water and evenly mixed. Then, the diluted PEI was added to a DNA aqueous solution, evenly mixed, incubated at room temperature for 15 min, and then used in transfection. Before the transfection, an original culture medium was sucked out, and 80 μL of a new culture medium was added, where a transfection dose of the DNA was 80 ng/well.

Negative control group: Only the 293T cells were used without transfection.

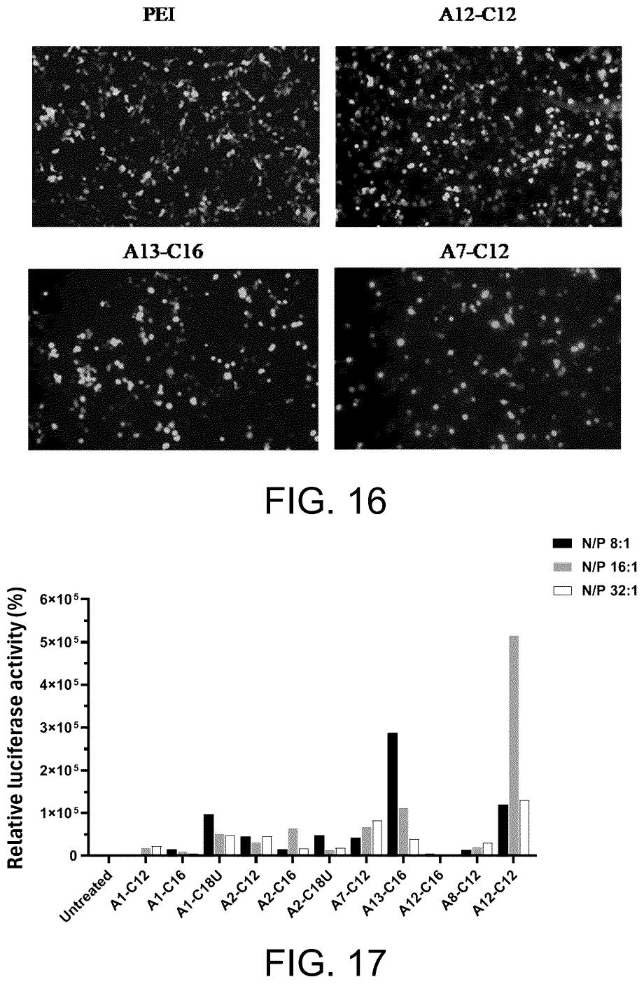

After the transfection, the expression of the green fluorescent protein was observed under a fluorescence microscope at 12 h, 24 h, 36 h and 48 h, respectively. After the transfection was performed for 48 h, the cells were lysed and subjected to centrifugation to remove cell debris and contents, a supernatant was taken, a firefly luciferase substrate was added, and an expression amount of firefly luciferase was determined so as to compare the plasmid transfection efficiency of the synthesized lipid compounds. Test results are as shown in FIG. 16 and FIG. 17. The transfection efficiency of the A12-C12 is equivalent to that of the commercial reagent PEI.

The embodiments of the present invention have been illustrated in detail above, but the present invention is not limited to the above embodiments, and various changes can also be made within the scope of knowledge acquired by persons of ordinary skill in the technical field without departing from the purpose of the present invention. Moreover, the embodiments of the present invention and features in the embodiments can be combined with each other without conflict.

1. A lipid compound or a pharmaceutically acceptable salt thereof, wherein the lipid compound is obtained by substituting all hydrogen atoms on an organic amine nitrogen with an R₁ group; the organic amine is selected from structures shown below: 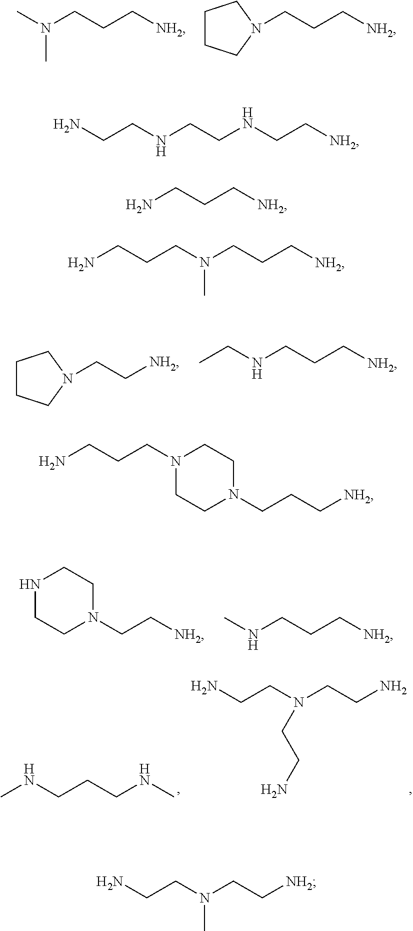
and the R₁ group has a structure shown in Formula (I): 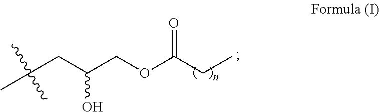
wherein, n is an integer selected from 6 to 16.

2. The lipid compound according to claim 1, wherein the organic amine is selected from structures shown below: 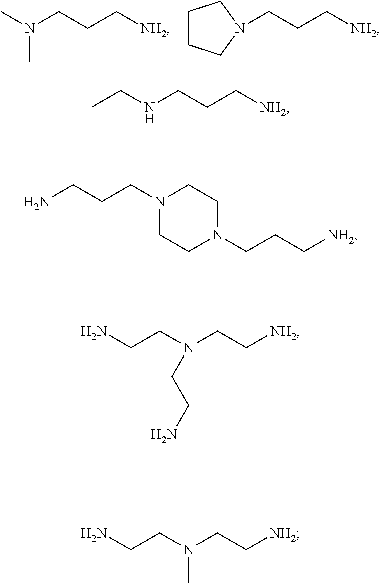
and further, the R₁ group is selected from structures shown below:

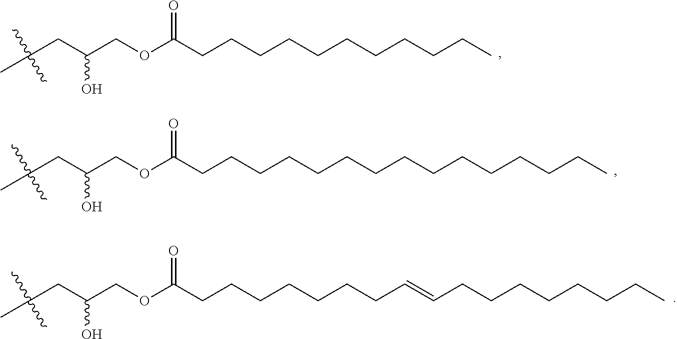 [SMILES: *CC(O)COC(=O)CCCCCCCCCCC ; *CC(O)COC(=O)CCCCCCCCCCCCCCC ; *CC(O)COC(=O)CCCCCCC/C=C/CCCCCCCC]

3. The lipid compound according to claim 1, wherein the lipid compound is selected from structures shown below:

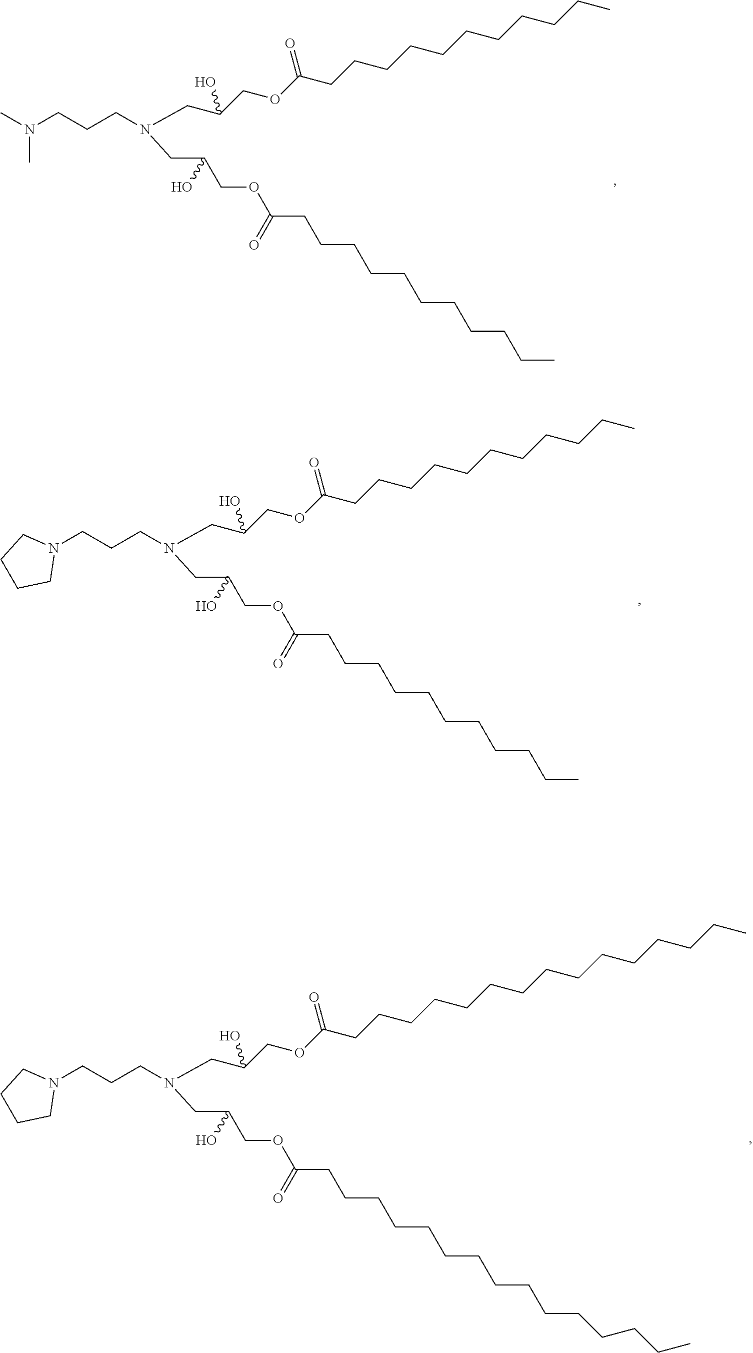 [SMILES: CCCCCCCCCCCC(=O)OCC(O)CN(CCCN(C)C)CC(O)COC(=O)CCCCCCCCCCC ; CCCCCCCCCCCC(=O)OCC(O)CN(CCCN1CCCC1)CC(O)COC(=O)CCCCCCCCCCC ; CCCCCCCCCCCCCCCC(=O)OCC(O)CN(CCCN1CCCC1)CC(O)COC(=O)CCCCCCCCCCCCCCC]

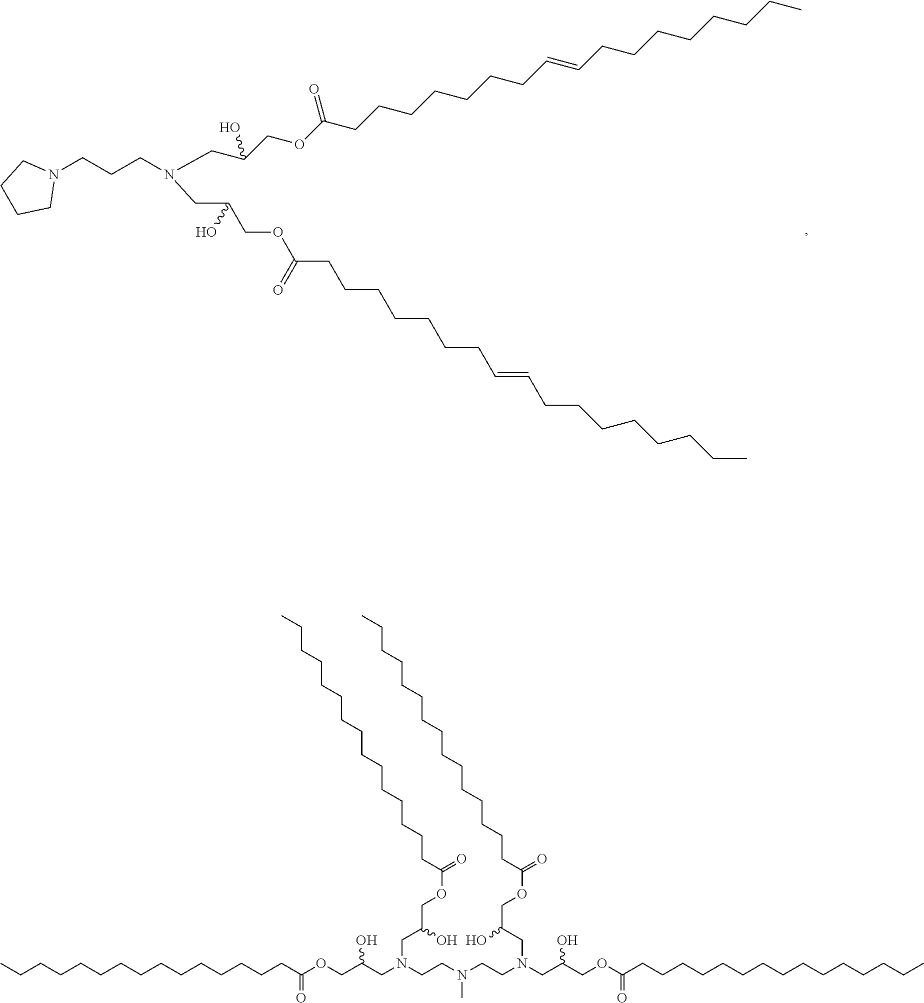

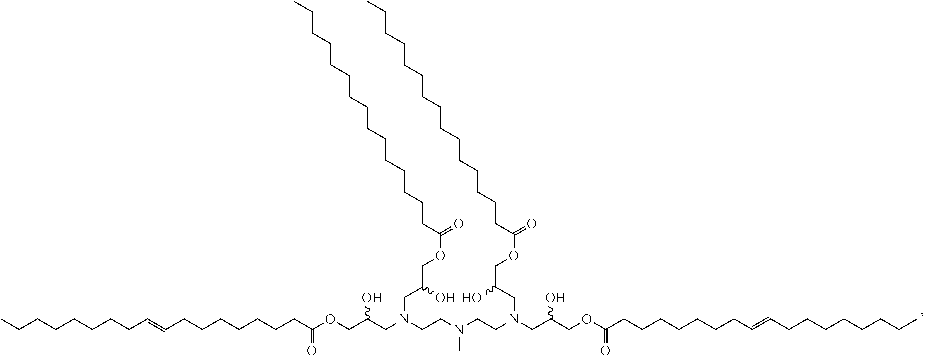 [SMILES: CCCCCCCC/C=C/CCCCCCCC(=O)OCC(O)CN(CCN(C)CCN(CC(O)COC(=O)CCCCCCC/C=C/CCCCCCCC)CC(O)COC(=O)CCCCCCCCCCCCCCC)CC(O)COC(=O)CCCCCCCCCCCCCCC]

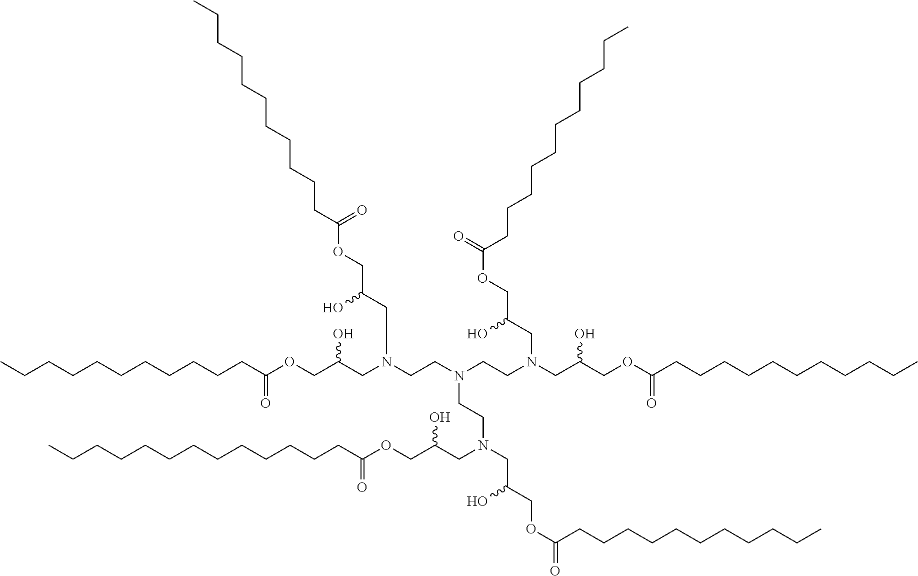 [SMILES: CCCCCCCCCCCCCC(=O)OCC(O)CN(CCN(CCN(CC(O)COC(=O)CCCCCCCCCCC)CC(O)COC(=O)CCCCCCCCCCC)CCN(CC(O)COC(=O)CCCCCCCCCCC)CC(O)COC(=O)CCCCCCCCCCC)CC(O)COC(=O)CCCCCCCCCCC]

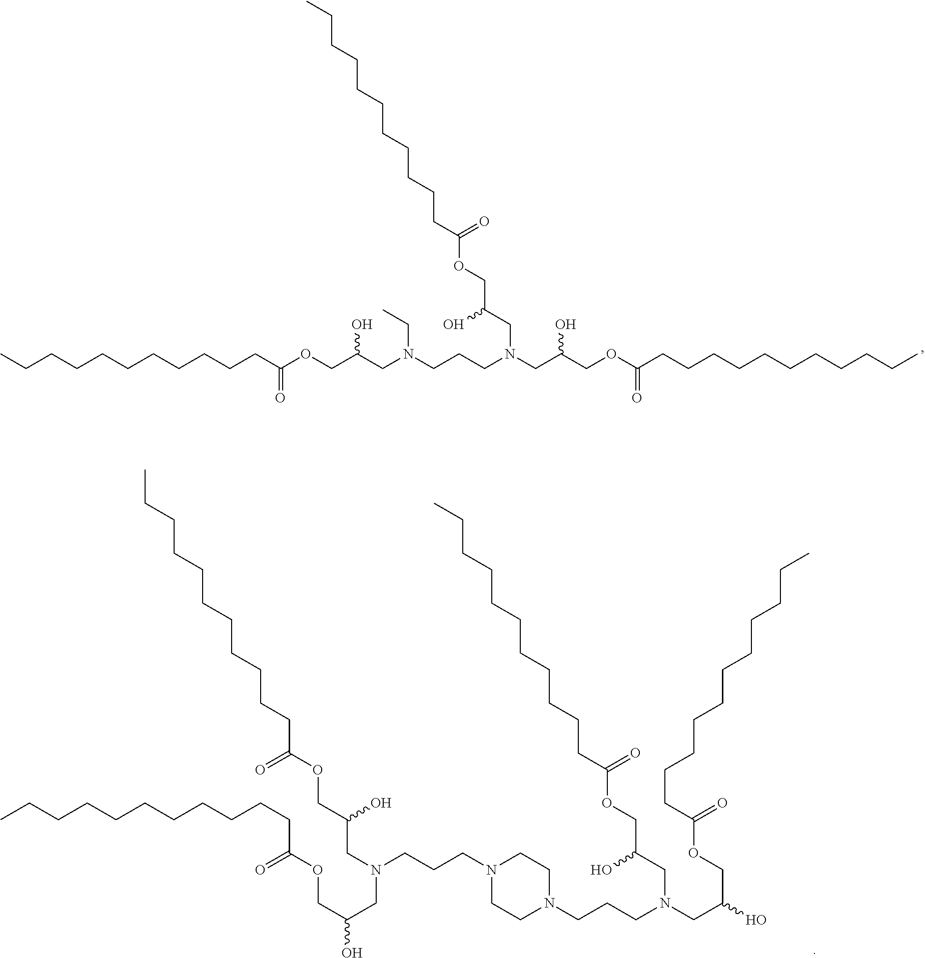

4. A composition, comprising the lipid compound or the pharmaceutically acceptable salt thereof according to claim 1; further, the composition further comprises other lipid compounds; and furthermore, the other lipid compounds comprise at least one of cholesterol, a phospholipid, and a polymer-conjugated lipid.

5. The composition according to claim 4, wherein the phospholipid comprises at least one of yolk lecithin, hydrogenated yolk lecithin, soy lecithin, hydrogenated soy lecithin, sphingomyelin, phosphatidyl ethanolamine, dimyristoyl phosphatidylcholine, dimyristoyl phosphatidylglycerol, dipalmitoyl phosphatidylcholine, distearoyl phosphatidylcholine, dioleyl phosphatidylcholine, and dilauryl phosphatidylcholine.

6. The composition according to claim 4, wherein the polymer-conjugated lipid comprises at least one of polyethylene glycol modified phosphatidyl ethanolamine, polyethylene glycol modified phosphatidic acid, polyethylene glycol modified ceramide, polyethylene glycol modified dialkylamine, polyethylene glycol modified diacylglycerol, and polyethylene glycol modified dialkylglycerol.

7. The composition according to claim 4, wherein a molar ratio of the lipid compound or the pharmaceutically acceptable salt thereof to the cholesterol is 1:1-9, preferably 1:1-5, more preferably 1:1-2;
further, a molar ratio of the lipid compound or the pharmaceutically acceptable salt thereof to the phospholipid is 1-10:1, preferably 1-5:1, more preferably 3-5:1;
and furthermore, a molar ratio of the lipid compound or the pharmaceutically acceptable salt thereof to the polymer-conjugated lipid is 10-50:1, preferably 10-20:1, more preferably 15-20:1.

8. The composition according to claim 4, wherein the composition further comprises an active pharmaceutical ingredient; further, the active pharmaceutical ingredient comprises at least one of a nucleic acid molecule, a polypeptide, a protein, and a small molecule compound; and furthermore, the nucleic acid molecule comprises at least one of an siRNA, an mRNA, an miRNA, an antisense RNA, CRISPR guide RNAs, a replicable RNA, a cyclic dinucleotide, poly IC, CpG ODN, and a plasmid DNA, preferably an siRNA.

9. The composition according to claim 8, wherein when the active pharmaceutical ingredient comprises the nucleic acid molecule, a nitrogen-phosphorus ratio of the lipid compound or the pharmaceutically acceptable salt thereof to the nucleic acid molecule is 1-50:1, preferably 1-40:1, more preferably 4-32:1.

10. Use of the lipid compound or the pharmaceutically acceptable salt thereof according to claim 1, genetic vaccines, polypeptide or protein drugs, and small molecule drugs.
